(19) United States
(12) Patent Application Publication
(10) Pub. No.: US 2011/0202114 A1
(43) Pub. Date: Aug. 18, 2011

(54) SYSTEM AND METHOD FOR TREATMENT OF LENS RELATED DISORDERS

(76) Inventors: Line Kessel, Roskilde (DK); Jesper Holm Lundeman, Smorum (DK)

(21) Appl. No.: 13/057,093

(22) PCT Filed: Jul. 16, 2009
(86) PCT No.: PCT/DK2009/050181

§ 371 Date: Apr. 28, 2011

(30) Foreign Application Priority Data
Aug. 8, 2008 (DK) PA200801084

(51) Int. Cl. A61N 5/06

(52) U.S. Cl. 607/88

(57) ABSTRACT

The present invention provides a means and a system for the prevention, treatment and/or amelioration of a disease and/or disorder which is either related to a lens of an eye and/or which may benefit from the treatment of said lens, the system comprising the exposure of said lens to radiation.

All patent and non-patent references cited in the application, are also hereby incorporated by reference in their entirety.

### FIELD OF INVENTION

The present invention relates to a system and use thereof for the prevention, treatment, or amelioration of diseases and disorders affecting the lens of the eye or which may benefit from the treatment of the lens.

### BACKGROUND OF INVENTION

Several widespread and debilitating diseases and disorders of the eye relate to the condition of the lens. The lens is a transparent structure in the eye that, along with the cornea, helps to refract light to be focused on the retina. By changing shape, the lens functions to change the focal distance of the eye so that it can focus on objects at various distances, allowing an image of the object of interest to be formed on the retina. This adjustment of the lens is known as accommodation, a feature which along with transparency is required for good vision.

Presbyopia (the age-induced need for reading glasses) is a very common condition affecting almost everybody from the age of 45 and onwards. According to the Helmholz theory, presbyopia is caused by increased stiffness of the lens with age which makes it harder to change the shape of the lens during accommodation and thus to focus the incoming light properly on to the retina.

Likewise, cataracts are a clouding or opacity that develops in the lens, varying in degree from slight to complete opacity and obstructing the passage of light. Cataracts may be congenital or develop with age; age-related cataract is the world-wide leading cause of blindness (Javit et al., 1996).

There are no means for preventing cataract and the only available treatment is invasive surgery wherein the clouded lens is replaced with an artificial lens (IOL). Presbyopia may be accommodated for by the use of reading glasses; however, many patients find the use of reading glasses very troublesome. All other commercially available products which may help restore accommodation are surgical and invasive. A single method has been approved by the FDA as a temporary cure for presbyopia: conductive keratoplasty; however, the effect only lasts for 3-6 months. As with cataract treatment, many of the methods of treating presbyopia are based on replacing the lens of the eye with an artificial lens. The artificial lenses have zones of different refractive indexes or diffraction, which in effect means that one eye is preserved for far-away vision and the other for close-up vision dependent on the lens implanted. This effect with zones of different refraction can also be achieved using the methods conventionally used for refractive surgery (presbyLASIK). A third principle, lentotomi, is based on cutting gliding planes inside the intact lens to allow for greater flexibility of the lens by the use of a femtosecond laser. The latter method is not commercially available.

There is a demand for an easy—as in non-invasive, permanent and pain-free cure for presbyopia, prevention, amelioration and treatment of cataracts and other diseases and disorders of the eye.

### SUMMARY OF INVENTION

Our invention, a simple light-based non-invasive method, provides a solution for the problem of lack of a non-invasive, permanent and pain-free cure for presbyopia, prevention and treatment of cataracts and other diseases and disorders of the eye. Apart from treating presbyopia, the light-based lens therapy herein described will delay the onset of cataract, thereby avoiding or postponing the need for cataract surgery by ten to possibly thirty years, a fact that is expected to lead to a major reduction, maybe 50% reduction, in the number of cataract surgeries carried out.

The unique aspect of the invention is that it is based on a simple, non-invasive light therapy where the age-related protein changes of the lens causing presbyopia and cataracts are reversed by radiation such as laser light. The advantages are:
1) the method is non-invasive, i.e. the usual surgical risks (infection, bleeding etc.) can be completely avoided.
2) The patient keeps his/her own lens which allows for a dynamic accommodation of the lens.
3) The method is relatively low-tech compared to e.g. lentotomi and presbyLASIK which means that the prices can be kept low allowing more patients to be treated and widespread global use of the present invention may be provided.

The treatment is based on photolysis of the aged altered proteins of the lens by use of a light source such as a laser, LED, or even sunlight. Surprisingly, the most efficient wavelengths include UV light and the lower wavelengths of the visible spectra of light. This is surprising, as it is the dogma amongst medical practitioners that UV and blue light are harmful to the eye and may be one of the causes of cataracts (Ophthalmology by M. Yanoff and J S Duker Published by Mosby International Ltd. 1999 and Adler's physiology of the eye—clinical application. Tenth edition. Editors: P L Kaufman and A Alm. Published by Mosby).

It is also disclosed herein that radiation of the lens by the system and method herein described has several effects on the lens and/or the proteins therein, one of these being the softening of the interior contents of the lens and a reduction/removal of the discoloration of the lens.

The present invention thus provides a method and a means for performing said method, the method being for the prevention, treatment or amelioration of a disease and/or disorder which is either related to the eye lens of an eye and/or which may benefit from the treatment of the lens, the method comprising the exposure of a lens to radiation of a wavelength substantially between 320 nm and 800 nm, thereby inducing changes in the lens and/or its constituents.

A first embodiment of the present invention regards a method of prevention, treatment and/or amelioration of a disease and/or disorder which is either related to a lens of an eye and/or which may benefit from the treatment of said lens, the method comprising the exposure of said lens to radiation of a wavelength substantially between 320 nm and 800 nm, thereby inducing changes in the lens and/or its constituents, comprising:
a) focusing a treatment light source into a selected part of the lens and/or its constituents collectively or selectively where treatment is intended to occur;
b) emitting light from said treatment light source;
c) measuring one or more types of radiation from at least the selected part of the lens and utilizing this measurement to decide to stop the treatment light source or to adjust at least one of the parameters: focus, intensity, wavelength, polarization, pulse length, repetition frequency, and pulse train length of said treatment light source,

whereby the photomanipulation is effectively monitored allowing for an accurate and optimum application of photo energy.

A second embodiment of the invention regards a method of prevention, treatment and/or amelioration of a disease and/or disorder which is either related to a lens of an eye and/or which may benefit from the treatment of said lens, the method comprising the exposure of said lens to radiation of a wavelength substantially between 320 nm and 800 nm, thereby inducing changes in the lens and/or its constituents, comprising:
a) focusing a treatment light source into a selected part of the lens and/or its constituents collectively or selectively where treatment is intended to occur;
b) emitting light from said treatment light source;
c) stopping said treatment light source after a predetermined period of time.

Thus, the first embodiment depends on feedback from measuring at least a part of the radiation emanating from the selected part of the lens. The feedback is used for varying one or more parameters of the radiation exposure in the treatment, i.e. exposure from the treatment light source. The second embodiment however, is more low-tech because the selected part of the lens is only exposed to radiation for a certain predetermined period of time. Thereby the method is simpler because it is independent of the feedback.

The predetermined period, i.e. the treatment period, is depending on one or more of the following parameters relating to the treatment light source, such as focus, intensity, wavelength, polarization, pulse length, repetition frequency, and pulse train. However, the age and type of the lens and the type and/or condition of the disease and/or the disorder may also be accounted for when the treatment period is determined.

A further embodiment provides an assessment phase after application of the said treatment light source where non-manipulative intensity is directed to the said selected part and measuring one or more types of radiation caused by the interaction between the said part and the said non-manipulative intensity and utilizing this measurement to decide to stop further treatment of said part or to resume treatment with or without adjustment of at least one of the following: focus, intensity, wavelength, polarization, pulse length, repetition frequency, pulse train length, scan velocity, size of scanned volume, scan repetitions, and scan pattern of said treatment light source.

Yet an embodiment is directed to a method wherein a procedure is performed comprising the following steps:

a) photomanipulating a selected part of a lens to be treated

b) detecting radiation from said selected part of the lens

c) gradually changing energy of said photomanipulation

d) registering when said radiation is within a defined threshold.

A further embodiment is to a method of treatment and/or amelioration of a presbyopic and/or cataractous disorder of a lens of an eye, the method comprising the exposure of said lens to radiation of a wavelength substantially between 350 nm and 550 nm, thereby inducing changes in the lens and/or its constituents, comprising;
a) focusing a treatment light source into a selected part of the lens and/or its constituents collectively or selectively where treatment is intended to occur, and
b) emitting light from the treatment light source, and
   c1) measuring one or more types of radiation at least from said selected part and utilizing this measurement to decide to stop the said treatment light source or to adjust at least one of the parameters: focus, intensity, wavelength, polarization, pulse length, repetition frequency, and pulse train length of said treatment light source, thereby photomanipulating the lens and/or its constituents thus treating and/or ameliorating said presbyopic and/or cataractous disorder, or
   c2) stopping said treatment light source after a predetermined period of time.

The methods of the present invention are carried out with a system/apparatus essentially as disclosed in the following embodiment: A system for prevention, treatment and/or amelioration of a disease and/or disorder which is either related to a lens of an eye and/or which may benefit from the treatment of said lens, said system comprising:
a) focusing means for focusing a treatment light beam of a wavelength substantially between 320 nm and 800 nm into a selected part of the lens and/or its constituents collectively or selectively where treatment is intended to occur, and
b) means for emitting and/or unblocking said treatment light beam, thereby inducing changes in the lens and/or its constituents, and
c1) means for stopping and/or blocking said treatment light beam after a predetermined period of time, and/or
d1) means for measuring one or more types of radiation from said selected part, and
d2) means for processing said one or more type of radiation from said selected part, and
d3) means for adjusting, based on at least part of the output of the means for processing, at least one of the parameters for said treatment light beam: focus, intensity, wavelength, polarization, pulse length, repetition frequency, and pulse train length of said treatment light beam.

An alternative embodiment regards a system where a procedure is performed comprising the following steps

a) photomanipulating a selected part of a lens to be treated

b) detecting radiation from said selected part of the lens

c) gradually changing energy and/or intensity of said photomanipulation

d) registering when said radiation is within a defined threshold.

Thus providing a system for the prevention, treatment and/or amelioration of a disease and/or disorder which is either related to a lens of an eye and/or which may benefit from the said treatment.

### BRIEF DESCRIPTION OF DRAWINGS

FIG. 1: Transmission spectra pre- and post treatment of young lens

FIG. 2: Transmission spectra pre- and post treatment of old lens

FIG. 3: Elasticity measures of treated vs. non-treated lenses

FIG. 4: Schematic drawing of the treatment system.

FIG. 5: Gray-scale photograph showing auto-fluorescence emitted from a lens (side-view) at the start of exposure to a 405 nm continuous wave laser (left) and at the end of laser exposure (right). Photographs were taken perpendicularly to the direction of the laser beam.

FIG. 6: Photographs of lens before and after exposure to a 405 nm cw.

FIG. 7: Transmission spectra pre- and post treatment with 405 nm cw laser.

FIG. 8: Transmission spectra pre- and post treatment with 532 nm cw laser

FIG. 9: Age-related decrease in light transmission.

FIG. 10: Model curves of transmission in the human lens at different ages relative to the transmission of a 10 year old lens.

FIG. 11: Changes in transmission in the human lens after irradiation with a 355 nm pulsed laser.

FIG. 12: Changes in transmission in the human lens after irradiation with a 400 nm femtosecond pulsed laser.

FIG. 13: Spectral transmission in vitro of human donors of different age.

FIG. 14: Lens transmission in vitro in selected spectral bands for 28 human lenses with regression curves describing the average reduction in transmission with age.

FIGS. 15A-15D: Illustrations of different treatment setups in cross-sections of the eye.

### DETAILED DESCRIPTION OF THE INVENTION

Definitions
Accommodation: The process by which the eye changes optical power to maintain a clear image (focus) on an object as it draws near the eye.
Astigmatism: A refractive defect of the eye in which incoming rays of different orientation are not focused into the same focal point resulting in a blurred image on the retina.
Cataract: Clouding that develops in the crystalline lens of the eye or in its envelope, varying in degree from slight to complete opacity and obstructing the passage of light.
Continuous wave: (herein abbreviated cw) is an electromagnetic wave carrying constant power.
Disease: An abnormal condition of an organism that impairs bodily functions, associated with specific symptoms and signs, typically causes discomfort and/or dysfunction. Herein used interchangeably with disorder.
Disorder: A functional abnormality or disturbance. Herein used interchangeably with disease.
Fluorescence: A luminescence that is mostly found as an optical phenomenon in cold bodies, in which the molecular absorption of a photon triggers the emission of another photon with a longer wavelength
Hyperopia: Also known as hypermetropia—a refractive defect of the eye in which collimated light produces image focus behind the retina when accommodation is relaxed.
Lens elasticity: Measured as deformation of the lens (flattening of lens surfaces and decreasing radius of curvature) as a response to increasing (or decreasing) centrifugal force.
Myopia: A refractive defect of the eye in which collimated light produces image focus in front of the retina when accommodation is relaxed.
Presbyopia: The condition where the eye exhibits a progressively diminished ability to focus on near objects with age.
Pulsed wave: An electromagnetic wave carrying non-constant power.
Refractive errors: An error in the focusing of light by the eye
Scattering: A general physical process whereby some forms of radiation, such as light, sound or moving particles, for example, are forced to deviate from a straight trajectory by one or more localized non-uniformities in the medium through which they pass.
Transmission: The fraction of incident light at a specified wavelength that passes through a sample.
Scheimpflug imaging: The Scheimpflug principle images the anterior eye with a camera at an angle to a slit-beam creating an optic section of the cornea and lens. It has been used e.g. for the assessment of keratoconus, cataract, intraocular lens tilt, decentration and corneal clearance and posterior subcapsular opacification after intraocular lens implantation. A popular commercially available Scheimpflug imaging system is the Pentacam. Scheimpflug imaging may provide an image of the lens in vivo, thus a Scheimpflug image may provide a measure of the light scattering by the lens and plurality of in vivo Scheimpflug lens images may provide a measure of change in light scattering in the lens

Diseases and Disorders of the Eye

The lens of the eye is comprised of densely packed lens fibrilles (cells) with very high protein content; approximately one third of the wet weight of the lens is protein (Glasser et al., 2001). These proteins undergo little or no renewal throughout life, the oldest proteins in the core of the lens date back to fetal life (Lynnerup et al., 2008). Lens transparency is highly dependent on the orderly spatial arrangement of the lens proteins (Benedek, 1971). Due to physical and chemical processes, the lens proteins are subjected to spontaneous posttranslational modification (Davies & Truscott, 2001). This results in protein aggregation which changes the arrangement of the proteins and leads to increased light absorption and scattering and less transmission of light through the lens. As there is almost no turn-over of proteins in the lens nucleus damaged proteins accumulate throughout the life-time of the individual.

The lens is located in the anterior segment of the eye. Anterior to the lens is the iris, which regulates the amount of light entering the eye. Posterior to the lens is the vitreous body and the fundus, which includes the retina, optic disc, macula, fovea, and posterior pole. The lens is suspended in place by the zonular fibers, which attach to the lens near its equatorial line and connect the lens to the ciliary body. The lens is flexible and its curvature is controlled by the ciliary muscles through the zonules. By changing the curvature of the lens, one can focus the eye on objects at different distances from it. This process is called accommodation. The lens has an ellipsoid, biconvex shape, and in the adult, the lens is typically 10 mm in diameter and has an axial length of 4 mm, though it is important to note that the size and shape can change due to accommodation and because the lens continues to grow throughout a person's lifetime.

As all light to the eye must not only pass through the lens, but also be refracted hereby in order for the light to be focused properly onto the retina, it is obviously of importance that the transmission and accommodation abilities of the lens are in order. This invention is directed generally to ameliorate, prevent and treat the consequences of ageing on the lens of the eye.

The lens, its capsule and its constituents are all aspects of the present invention. These elements may be treated collectively and/or selectively. Preferably, it is the lens and its protein constituents that are treated with the herein disclosed method with the herein described system.

Presbyopia

For a clear image to be formed on the retina while viewing objects at different distances requires accommodation, i.e. a change in refractive power of the lens. As stated above, the refractive power of the lens is changed by changing the lens curvature by increasing or decreasing the tension of the muscles in the ciliary body whereby the zonules exert a stretching or relaxing force on the lens. The mechanisms behind age-related loss of accommodation, presbyopia, are not known with certainty; however, research supports the theory of loss of elasticity of the crystalline lens, although changes in the lens's curvature from continual growth and loss of power of the ciliary muscles (the muscles that bend and straighten the lens) have also been postulated as its cause.

The increased lens stiffness accompanies an increased yellow-brownish discoloration of the lens and increased scattering of light. This is believed to be related to denaturation of the lens proteins by glycation and other processes which ultimately results in large protein aggregates (Monnier & Cerami, 1981). Breaking the chemical bonds that hold the large protein aggregates together restores the initial state of the lens. The denaturation products are susceptible to photolysis by ultraviolet radiation (Kessel et al., 2005). However, ultraviolet radiation is toxic to the retina and cornea. The studies herein disclosed (see Examples) demonstrate that the same positive photolysis effects can be achieved using less harmful light of longer wavelengths.

The ageing processes of the lens leads to biochemical changes on the lens proteins which result in products capable of emission of auto-fluorescence (Monnier & Cerami, 1981). The auto-fluorescence can be used as an internal reference for dosimetry of the treatment herein disclosed.

To find a cure for presbyopia and other diseases, the effect of irradiating the lens with monochromatic laser light of different wavelengths on the yellow coloration and light transmission was investigated in human donor lenses. A significant increase in transmission from 400-550 nm after exposure to monochromatic laser light at 400 and 405 nm corresponding to the transmission of a 15 to 20 year younger lens was found; please see the Examples for details. Initial findings found a maximum effect which was reached at an irradiance dose of 6.8 kJ/cm². Later findings found a clinical significant effect at much lower irradiance doses (see Examples 1-3). Exposure to laser light at 532 nm leads to a small increase in transmission from 440-540 nm.

It is an object of the present invention to prevent, treat, and/or ameliorate presbyopia by the exposure of a lens to radiation, thereby inducing changes in the lens and/or its constituents. Particularly it is an object of the present invention to increase the accommodation ability of the lens by the administered treatment.

Cataract

Cataract is defined as any opacity of the lens of the eye which impairs visual function and is closely related to an increased absorption and scattering of light. Early in the development of age-related cataract the power of the lens may be increased, causing near-sightedness (myopia), and the gradual yellowing and opacification of the lens may reduce the perception of blue colors. Cataracts typically progress slowly to cause vision loss and are potentially blinding if untreated.

Nuclear sclerosis is an early stage cataract caused by compression of older lens fibers in the nucleus by new fiber formation. The more dense construction of the nucleus causes it to scatter light rather than to allow unhindered transmission of the light through the lens.

The yellow coloration of the lens accompanying cataract is, as stated above, believed to be caused by the formation of covalent cross-links and aggregation of degraded proteins in the lens. Molecular cross-links and other types of degradation disrupt the optical and mechanical properties of the lens. The cross-links may be sulphur bridges occurring between and/or within the proteins of the lens. The fluorescence of cyclic molecular components of the cross-links is early evidence of this process.

It is an object of the present invention to prevent, treat, and/or ameliorate cataract by the exposure of a lens to radiation, thereby inducing changes in the lens and/or its constituents. Particularly it is an object of the present invention to increase the transmission of the lens by the administered treatment.

Myopia

Myopia or near-sightedness is due to a refractive defect of the eye in which collimated light produces image focus in front of the retina when accommodation is relaxed. In other words, the eyeball is too long, or the cornea is too steep, so images are focused in the vitreous inside the eye rather than on the retina at the back of the eye. This causes nearby objects to be seen clearly but distant objects to appear blurred.

Myopia has several causes and forms. It may be caused by an increase in the eye's axial length or the condition of the refractive elements of the eye and may present itself as simple myopia (an eye that is too long for its optical power) which is the most common form; degenerative myopia, characterized by marked fundus changes; nocturnal myopia (also known as night myopia) a condition in which the eye has a greater difficulty seeing in low illumination areas; pseudomyopia due to spasms of the ciliary muscle; induced myopia or acquired myopia, which results from exposure to various pharmaceuticals, increases in glucose levels, or nuclear sclerosis amongst others.

By altering the shape of the lens it is possible to alter the refraction of a myopic eye allowing incoming light to be focused on the retina and thus alleviating the myopic condition.

It is an aspect of the present invention to treat and/or ameliorate myopia by the exposure of a lens to radiation, thereby inducing changes in the lens and/or its constituents. It is of importance that the treatment alters the refraction of the lens allowing proper focus.

Hyperopia

Is as myopia a refractive defect of the eye, the cause and effect are however different: the incoming light is focused behind the retina when accommodation is relaxed. This may occur when the eyeball is too short or when the lens cannot become round enough, causing inability to focus on near objects.

As with myopia, altering the shape of the lens makes it possible to alter the refraction of a hyperopic eye allowing incoming light to be focused on the retina and thus alleviating the hyperopic condition. It is thus an aspect of the present invention to treat, and/or ameliorate hyperopia by the exposure of a lens to radiation, thereby inducing changes in the lens and/or its constituents. It is of importance that the treatment alters the refraction of the lens allowing proper focus.

Astigmatism

Astigmatism is an optical defect, whereby vision is blurred due to the inability of the optics of the eye to focus a point object into a sharp focused image on the retina. This may be due to an irregular or toric curvature of the cornea or lens. There are two types of astigmatism, regular and irregular. Irregular astigmatism is often caused by a corneal scar or scattering in the crystalline lens and cannot be corrected by standard spectacle lenses. Regular astigmatism arising from either the cornea or crystalline lens may at times be corrected by use of spectacle lenses, but as with presbyopia, a permanent solution is preferable.

It is an object of the present invention to provide the means and a method with which to treat, and/or ameliorate astigmatism caused by conditions of the lens of the eye by the exposure of the lens to radiation, thereby inducing changes in the lens and/or its constituents. It is of importance that the treatment alters the refraction of the lens allowing proper focus.

Mental Diseases

A mental disorder or mental illness is a psychological or behavioral pattern that occurs in an individual and is thought to cause distress or disability that is not expected as part of normal development or culture. For example a depressed mood is often reported as feeling sad, helpless, and hopeless.

The young lens is highly transparent to all visible wavelengths but transmission decreases drastically after the age of thirty. Though the accumulation of yellow chromophores is responsible for the preferential loss of light transmission in the blue end of the spectrum it is not in itself a cause of loss of visual function but it reduces the ability to perceive and distinguish shades of blue. Decreased transmission of blue light by the aged lens may be responsible for the increased risk of depression and sleep disorders seen in the elderly population through depressed activation of melanopsin. Melanopsin is a photosensitive pigment that is important for circadian photo-entrainment by stimulating melatonin secretion by the pineal gland. It is expressed in a small subset of retinal ganglion cells. It has an absorption maximum near 480 nm and it is active only in bright light. Thus, dim light and a reduced transmission of blue light has a major impact on the circadian pace, sleep disorders and mental mood.

It is an object of the present invention to provide the means and a method with which to treat, and/or ameliorate mental illness, such as depression, winter depression, depressive mood, caused by conditions, e.g. aging, of the lens of the eye by the exposure of the lens to radiation, thereby inducing changes in the lens and/or its constituents. It is of importance that the treatment alters the transmittance of the lens, in particular to increase transmittance of light in the blue region. Thereby the means and methods according to the invention may be useful in various applications such as retinal light dosimetry during therapeutic light exposure or in psychophysical examinations and experimental phototherapy of winter depression.

Intraocular Lens (IOL)

As will be demonstrated in the following increasing age is associated with a gradual decrease in the transmission of all wavelengths with an accelerated loss of transmission for shorter wavelengths. Empirical formulas describing the age-related loss of transmission will be presented for each of the spectral colors and it will be shown that the transmission of light through the human lens decreases with age in a highly predictable manner. A monotonous decrease in transmission from the red to the blue end of the spectrum with age will be demonstrated.

The mathematical description of this process may be of value for the design of implant lenses with characteristics that mimic those of a human lens of a given age and in psychophysical studies of melanopsin activation. Thus, a further embodiment of the invention regards a system and a method for designing an IOL to be implanted in a patient's eye, wherein the IOL design is individually adapted to the age of the patient, thereby accounting for the retinoprotective effects of an aged human lens, in particular in the blue spectral range. The IOL design preferably accounts for the average reduction in transmission with age in one or more of the following spectra bands: violet (400-449 nm), blue (450-489 nm), green (490-559 nm), yellow (560-589 nm), orange (590-629 nm), red (630-699 nm), and infra-red (700-800 nm). The reduction in transmission for the spectral bands may be accounted for by using one or more of the regression parameters of Table 1 below.

Thus, the quantitative model of spectral light transmission in the human lens may be of value in providing new quantitative measures of lens transmission that may assist in the study of the activation of the melanopsin and the retinohypothalamic tract and in the design of new IOL that mimic the retinoprotective effects of the aged human lens without compromising circadian entrainment.

System (Apparatus)

The present invention provides the means and methods for the prevention, treatment and/or amelioration of diseases and/or disorders which are either related to the lens of an eye and/or which may benefit from the treatment of the lens. The method comprises the exposure of a lens in need of treatment to radiation, thereby inducing changes in said lens and/or its constituents. The means provide a system for performing the method upon the lens to be treated, and means such as software or other propagated signal which enables the system to perform the method of above.

Thus, an object of the present invention is a system which may be used for the prevention, treatment and/or amelioration of diseases and/or disorders which are either related to the lens of an eye and/or which may benefit from the treatment of the lens, by exposing said lens to radiation, thereby inducing changes in the lens and/or its constituents.

Generally, the system comprises an optical system for focusing a light source, i.e. focusing the light beam of a light source, and preferably also means for adjusting the light beam. The means for adjusting may simply be means for blocking/unblocking the light beam, but may also be more advanced means for controlling the light beam. One or more light sources for emitting one or more light beams may also be part of the system according to the invention. Further and more advanced embodiments of the invention comprises a 3D scanner, a computer interface, and means for detecting fluorescence and/or scattering emitted from a lens prior to, during and after treatment. The system may furthermore comprise a secondary light source for the monitoring of treatment parameters, means for tracking the movement of the lens/eye undergoing treatment and/or means for fixating the lens/eye undergoing treatment.

Treatment Control Parameters

When irradiating a lens, if precautions are not made several types of damages may occur. These include white, opaque lesions on the surface of the lens facing the laser if, for instance laser pulse energy is too high, and/or the formation of gas blisters or bubbles within the lens. A study has shown that exposure to high pulse energy UVA and UVB in young animal lenses leads to protein polymerization and increased optical density (Dillon et al., 1989). Lens transparency is closely related to lens protein-protein interaction (Ponce et al., 2006) and protein polymerization is thus expected to lead to lens opacities (Benedek, 1971).

It is an object of the present invention to provide a safe means of treating the herein disclosed diseases and disorders. In one embodiment this is accomplished by measuring several parameters prior to, during and/or after the treatment is administered. These parameters include, but are not limited to: Light scattering, Transmission, Accommodation and Fluorescence. In another embodiment the treatment is merely stopped after a predetermined period of time.

The lens may be characterized in advance by comparing to a color reference, e.g. according to the Lens Opacities Classification System (Chylak et al.), that has been developed to grade cataract severity. Thus, by providing an image of the lens prior to treatment the lens may be graded and the treatment may be planned in advance. Thus, the treatment period may be provided based on one or more lens images and a grading according to a lens classification system. The lens may also be graded according to a lens classification system during the treatment and/or after the treatment according to the invention.

Light scattering may be measured by shining a light onto the lens and measuring the amount and degree of the reflected light, e.g. by means of Scheimpflug imaging.

Prior to treatment Scheimpflug imaging may provide a measure of the severity of a disease or disorder, during treatment Scheimpflug imaging may provide a measure of the progress of the treatment and after treatment Scheimpflug imaging may provide a measure of the effect of the treatment.

The transmission properties of the lens reflect the degree of coloration of the lens. This parameter may be measured in vivo by registering the amount of light reflected from the lens.

In vitro, the accommodation ability of the lens may be measured as described in example 3 by measuring the effect of applying a centrifugal force to the lens and following the shape change. In vivo, the accommodation ability may be measured by a functional visual test such as the minus method (Anderson H A 2008) or the Donders push-up test.

The intrinsic autofluorescence of the lens is an indicator of biological lens age and may be used in conjunction with the treatment process to diagnose, monitor and adjust treatment parameters.

Many of these treatment control parameters are measurable by radiation and thus it is an aspect of the present invention that the system is capable of detecting the various types of radiation as well adapt the treatment based on these measurements.

The radiation that is a measure of the treatment control parameters comprises at least one of the following: fluorescence (detection of broad-band emission of light from the target, at longer wavelength than that of the incoming light), scatter (detection of light emitted from the target at the wavelength of the incoming light), Raman scatter (detection of narrow-band emission of light from the target, at longer or shorter wavelength than that of the incoming light), reflection (specular reflection of the incoming light), phosphorescence (detection of broad-band emission of light from the target, at longer wavelength than that of the incoming light and with a delay of more than 100 nanoseconds), and bremsstrahlung (detection of broad-band emission of light from the target, at both longer and shorter wavelengths than that of the incoming light). Incoming light may be the light provided by a secondary light source probing the lens. In the case of Scheimpflug imaging the incoming light is provided by the light source that is part of the Scheimpflug imaging system. Specifically, it may be useful to measure the spectral distribution of the radiation and thereby utilizing that the spectral radiation is often specific to its molecular origin. Similarly, different time constants, such as relaxation time, may reveal properties of the treatment site. In one embodiment of the invention it is therefore preferred to analyze the measurement of the radiation by temporally resolved analysis.

One object of the present invention to provide a system that not only measures the above parameters, but also adjusts the administered treatment accordingly both prior to and during the procedure. This is accomplished by one or more feedback mechanisms.

Feedback

An important aspect of the system relates to the possibility for monitoring and adapting the treatment, this is herein described as feedback/feedback mechanisms. By performing measurements of the various treatment parameters as well as determining the size, shape, thickness and other aspects of the lens, it is possible to adapt the treatment to each individual lens to be treated. Furthermore, by scanning separate sections of the lens prior to and during treatment, it is possible to adapt and adjust the treatment to each individual section of the lens thereby providing the best possible treatment. A goal for the treatment may be defined prior to the treatment and may be used for determining the treatment period.

Any of the treatment control parameters and/or the radiation characterizing these can be used to monitor the state of the lens prior to and during induction of photochemical reactions by the treatment light beam. At least one measurable treatment control parameter may arise due to the radiation stemming from the treatment light beam. Thereby, the characteristics of the radiation, the analysis of which is used to adjust the treatment light beam will depend directly on the treatment light beam. However, it may also be that the at least one treatment control parameter is initiated and/or followed by a secondary source of radiation, such as a laser or LED, and/or by monitoring by means of e.g. Scheimpflug imaging. Thereby, the lens or selected part of same may e.g. be manipulated at one wavelength but probed for characterizing radiation/treatment control parameter using a different wavelength.

A preferred embodiment of the method according to the invention further comprises an initialization phase, where non-manipulative intensity is directed to a selected part of the lens and one or more types of radiation, caused by the interaction between said selected part and the non-manipulative intensity, are measured and utilizing this measurement to decide not to photomanipulate the selected part or decide to proceed with photomanipulation. Thereby, the suitability of the selected part for photomanipulation may be assessed. Furthermore, it is preferred that the initialization phase is further utilized to adjust at least one of the following features of the treatment laser beam: focus, intensity, wavelength, polarization, pulse length, repetition frequency, pulse train length, scan velocity, size of scanned volume, scan repetitions, beam polarization and direction. Thereby the setting of the parameters for the photomanipulation is pre-optimized so that the probability of undesired effect arising from the photomanipulation is minimized.

A second preferred embodiment of the method according to the invention comprises an assessment phase after application of the treatment light beam where non-manipulative intensity is directed to a selected part of the lens and measuring one or more types of radiation caused by the interaction between the selected part and the non-manipulative intensity and utilizing this measurement to decide to stop further treatment of the selected part or to resume treatment with or without adjustment of at least one of the following: focus, intensity, wavelength, pulse length, repetition frequency, pulse train length, polarization, size of scanned volume, scan repetitions, beam polarization and direction, and scan pattern of said treatment light beam, which might be a treatment laser beam. Thereby, it may be verified if sufficient photomanipulation has occurred or if it has not the results of the said assessment may be used to optimize further photomanipulation.

In a preferred method according to the invention the measurement involves determining the optical signature of a selected part of the lens comprising at least one of the following: transient characteristics arising as an effect of the treatment pulse or any characteristic that can be recorded using steady-state or time-resolved spectroscopy (such as a change in color and absorption), Raman spectroscopy (such as a change in Stoke's shift and Raman scatter intensity), photon-correlation spectroscopy (such as a change in apparent molecular weight, rigidity, and composition), light scattering in the lens (measuring the changes in how the lens scatters light, e.g. by means of Scheimpflug imaging), fluorescence spectroscopy (a reduction, increase, spectral shift or other change in lens fluorescence) and/or phosphorescence spectroscopy (a reduction, increase, spectral shift or other change in lens phosphorescence).

In one embodiment of the invention the measurement involves detection of acoustic effects recorded using non-contact sensor(s) and/or an acoustic sensor placed in direct contact or indirect contact with the eye or adjacent tissue. Non-contact acoustic sensors are well-known in the art such a microphones or laser interferometry of laser light reflected off a surface.

Acoustic effects in conjunction with photomanipulation of the lens arise from the forming and especially the collapse of gas blisters and from other types of interaction between light and tissue. Accordingly, including the acoustic sensor thereby provides for a primary detection of the formation of gas blisters forming as a result of the photomanipulation. With a direct detection it is then possible to positively verify whether or not gas blistering has taken place, and/or to stop further photomanipulation if it does.

In a most preferred method according to the invention, the measurement, analysis and adjustment form a feed-back loop, so the steps of measuring, analyzing and adjusting occur substantially continuously. It is furthermore preferred that the feed-back loop operates substantially in real-time. It is preferred that the measurement, processing of resulting data, said adjustment(s), and renewed photomanipulation of the selected part of the lens occurs at least within substantially 0.1 second or a shorter time period which is substantially smaller than the spontaneous movements of the eyes (saccades) and preferably shorter than 0.01 second. Within a response time of this order micro movements of the eye may be ignored so that the site from which radiation is measured corresponds to the site subsequently irradiated.

It falls within the scope of the present invention that the measurement, processing of resulting data, said adjustment(s), and renewed photomanipulation of the selected part of the lens occurs within an interval of at least 10 seconds, preferably within 1 second, more preferably within 0.1 seconds and most preferably at least within substantially 0.1 second or a shorter time period such as 0.01 seconds.

The feedback mechanisms are essentially computer control means, such as algorithms, computer data signals, and/or propagated signals. These may be stored on a computer program product or be available online over e.g. the internet or a closed circuit.

Adaptive Optics

It is preferred that the at least one treatment light beam and/or any secondary source of radiation is focused using adaptive optics. In ophthalmology, adaptive optics may be applied to compensate for aberrations due to imperfections in the eye tissue, so that the focus of the treatment light beam is optimized. It is preferable that the adaptive optics further comprises the use of a deformable mirror. Furthermore, it is preferred that the adaptive optics further comprises the use of a Hartmann-Schack sensor. Furthermore it is preferred that the adaptive optics further comprises the uses of liquid crystal phase plates. While single-shot adjustment using adaptive optics is possible it may for some applications be preferable that the adaptive optics form a feedback loop where adjustment and measurement of the result is an iterative process. The adaptive optics may be guided by a light source dedicated to this purpose but it is preferably guided by reflection or other radiation caused by the treatment light beam or a secondary source of radiation.

Goal Related Feedback

In the above described feedback system one or more of several physical goals may be considered in the programming of the feedback system. These goals are herein termed predefined treatment goals. Accordingly, the feedback system may be programmed to observe specific physical properties and adjust the light source and/or the decision to progress or stop the treatment based on this property. In a preferred embodiment of the invention, such monitoring may be performed by the following steps:
photomanipulating a selected part of a lens
detecting intensity of radiation, spectral distribution, beam deflection, scattering, reflection, transmission and/or polarization at least from the selected part of the lens
gradually changing treatment parameters of said selected photomanipulated part of the lens
registering when one or more of said detected parameters fit the predefined treatment goal.

Similarly, in another preferred embodiment of the invention the efficiency of the treatment is investigated by measurement of radiation due to a non-manipulative intensity directed to the selected part of the lens. Finally, such investigation of the efficiency may be performed or supplemented by comparing values of radiation obtained prior to treatment with the data obtained from the verification

In general the goal of the feedback system may be to have said radiation increase, decrease, appear, disappear or have a suitable level. Among the preferred goals of the adjustment of the treatment light beam according to the present are adjustment to obtain bleaching, color change, deaggregation of lens components, depolymerization of lens proteins or other constituents of the lens, or resolubilization of lens proteins or other constituents of the lens. This is preferably carried out while avoiding or minimizing cavitation, mechanical effects, acoustic effects, and/or thermal effects on molecules, components, or cells that do not form a target for treatment or are outside said selected area. In the effort to avoid the said event the system may monitor the same or other radiation as the radiation(s) used to determine achievement of the said goal.

Among other preferred goals of the adjustment according to the invention are adjusting the treatment light beam to obtain molecular cleavage of specific molecules or macromolecular adducts, for instance lens proteins or lens protein cross-links, without damage to healthy lens proteins, cell membranes or other healthy components of the lens, and further avoiding or minimizing cavitation, mechanical effects, acoustic effects, and/or thermal effects on molecules, components, or cells that do not form a target for treatment or are outside said selected area. More specifically said radiation may be fluorescence and the system adjusts to simultaneously minimizing or preventing an increase in scattering.

Treatment Radiation Characteristics

Dogma dictates that photonic excitation of specific molecular constituents of the human eye using blue light or ultraviolet is problematic, because the energetic photons can cause damage to the cornea and the living layers of the lens. Additional problems include retinotoxicity and poor penetration of cataractous lenses. The studies underlying the present invention surprisingly have found that it is not only possible to use short wavelength light for the treatment, prevention and/or amelioration of diseases and disorders of attaining to the lens of the eye, but that using said light furthermore is advantageous.

The advantages include: That short wavelength light holds sufficient energy to provide the desired effects of reversing e.g. age-related stiffness and opacities of the lens. Simultaneously, the aged human lens absorbs light of short wavelength; hereby the light enters the lens, but does not penetrate the lens and thus damage the retina. Furthermore, light of short wavelengths, especially blue light, diverge less than e.g. infrared light. This is of consequence to the numerical aperture for lenses etc. of the system which due to less divergence can be minimized in size—making the system smaller and cheaper to produce. Monochromatic light of short wavelengths can furthermore be focused to an area approx. one fourth of the area of i.e. the area covered by an 800 nm laser. Thus the desired effects are obtained all the while the eye is protected from damage with a smaller cheaper and better performing system compared to those known in the prior art.

The present invention regards the emission of light from any source capable hereof. The light may be emitted from one, two, three or more treatment light sources, just as there may be one, two three or more secondary beams for the purpose of verifying the treatment. The emitted light may be continuous wave light (herein denoted cw) or pulsed light. A plurality of treatment light beams may have the same or different wavelengths of light and may be either cw light only, pulsed light only or a combination of cw and pulsed light. The at least one treatment light source may emit monochromatic and/or polychromatic light and there may be emitted light of the same, different and/or a plurality of wavelengths simultaneously and/or time and/or spatially displaced from each other.

If polychromatic light is to be employed in the system it is an aspect of the invention to provide a means for altering the polarization of the emitted photons and/or their direction. The strength of a polychromatic source and the means and methods of modulating the light are all known to a person skilled in the art.

As can be ascertained from the Examples, very similar results were produced no matter whether a pulsed or a cw system was used. Therefore both manners of irradiation are aspects of the present invention.

In one embodiment at least one treatment light beam, such as at least one treatment laser beam, is mono- and/or polychromatic light provided as cw and/or pulsed light at a wavelength between 260 nm and 1400 nm, such as between 280 and 1200 nm, such as between 300 and 1100 nm, such as 320 and 1000 nm, such as 340 and 900 nm, such as between 350 and 800 nm.

Preferably, the at least one treatment light beam is monochromatic light emitted as cw and/or pulsed light of a wavelength between 300 and 800 nm, such as 310 and 750 nm, such as 320 and 700 nm, such as 330 and 650 nm, such as 340 and 600 nm, such as 350 and 550 nm, such as 360 and 500 nm, such as 370 and 480 nm, such as 380 and 460 nm, such as 390 and 440 nm, such as 400 and 420 nm, such as 410 and 480 nm, such as 430 and 460 nm, such as 435 and 455 nm, such as 440 and 450 nm, such as 442 and 448 nm. Also it is preferred that the at least one treatment light beam is light of between 315 and 600 nm, such as 325 and 575 nm, such as 350 and 555 nm, such as 380 and 525 nm, such as 400 to 500 nm.

A treatment light beam around 445 nm may be of particular interest. Studies (Gorgets, 1995) have shown that damage sensitivity in rat retina increases manifold from visible to ultraviolet wavelengths. Exposure of the retina to radiation below 440 nm caused cell damage to photoreceptors in the retina, whereas radiation above 470 nm did not. Thus, it seems that a treatment light beam around 445 nm is a good compromise between providing an efficient photomanipulation of the lens without causing damage to the retina. Good quality low price light sources, e.g. semiconductor lasers, around 445 nm are also widely available.

Most preferably the at least one treatment light beam is monochromatic light of a wavelength as provided by any standard laser known to a person skilled in the art, such as, but not limited to: 387 nm, 395 nm, 405 nm, 415 nm, 430 nm, 441.6 nm, 442 nm, 445 nm, 450 nm, 458 nm, 473 nm, 488 nm, 514 nm and/or 532 nm.

Continuous Wave Light

One aspect of the present invention relates to the use of continuous wave (cw) light for the treatment, prevention and or amelioration of age-related and other diseases and disorders of the eye that are related to the lens or may benefit from treatment of the lens. Thus, in one embodiment the at least one treatment light beam is continuous wave light of a wavelength between 300 and 800 nm, such as 315 and 760 nm, such as 325 and 720 nm, such as 335 and 680 nm, such as 345 and 640 nm, such as 355 and 600 nm, such as 365 and 560 nm, such as 375 and 520 nm, such as 385 and 480 nm, such as 390 and 440 nm, such as 395 and 430 nm, such as 400 and 420 nm, such as 410 and 480 nm, such as 430 and 460 nm, such as 435 and 455 nm, such as 440 and 450 nm, such as 442 and 448 nm. Also it is preferred that the at least one treatment light beam is light of between 315 and 600 nm, such as 325 and 575 nm, such as 350 and 555 nm, such as 380 and 525 nm. A very preferred embodiment comprises a system capable of emitting cw light of a wavelength between, and including the endpoint of 400 nm to 500 nm.

The power of the treatment light beam may be between 0.1 and 1 W, such as 0.1 and 1 mW, such as 1 and 10 mW, such as 10 and 40 mW, such as 10 and 40 mW, such as 40 and 100 mW, such as 100 and 150 mW, such as 150 and 200 mW, such as 200 and 300 mW, such as 300 and 500 mW, such as 500 mW and 1 W.

However, what really matters in terms of treatment and impact to the lens is the power of the treatment light beam per area, i.e. the intensity. The intensity of the treatment light beam will depend of the focus and where it is measured. The most relevant location of measuring the intensity of the treatment light beam will be right in front of the cornea, where the focus of the beam also typically be most intense. In the preferred embodiment of the invention the intensity of the treatment light beam is around 1 to 10 mW/mm² corresponding to 10⁻⁸ to 10⁻⁸ W/m². However, the intensity of the treatment light beam may be between 0.01 mW/mm² and 1000 mW/mm², such as between 0.01 mW/mm² and 0.1 mW/mm², such as between 0.1 mW/mm² and 1 mW/mm², such as between 1 mW/mm² and 10 mW/mm², such as between 10 mW/mm² and 50 mW/mm², such as between 50 mW/mm² and 100 mW/mm², such as between 100 mW/mm² and 500 mW/mm², such as between 500 mW/mm² and 1000 mW/mm².

Most preferably the at least one treatment light beam is monochromatic cw light of a wavelength as provided by any standard laser or LED known to a person skilled in the art, such as, but not limited to: 387 nm, 395 nm, 405 nm, 415 nm, 430 nm, 442 nm, 445 nm, 450 nm, 458 nm, 473 nm, 488 nm, 514 nm and/or 532 nm.

Pulsed Light

In another embodiment the at least one treatment light source emits pulsed light of a wavelength between 300 and 800 nm, such as 315 and 760 nm, such as 325 and 720 nm, such as 335 and 680 nm, such as 345 and 640 nm, such as 355 and 600 nm, such as 365 and 560 nm, such as 375 and 520 nm, such as 385 and 480 nm, such as 390 and 440 nm, such as 395 and 430 nm, such as 400 and 420 nm, such as 410 and 480 nm, such as 430 and 460 nm, such as 435 and 455 nm, such as 440 and 450 nm, such as 442 and 448 nm. Also it is preferred that the at least one treatment light source emits light of between 315 and 600 nm, such as 325 and 575 nm, such as 350 and 555 nm, such as 380 and 525 nm. A very preferred embodiment comprises a system capable of emitting pulsed light of a wavelength between, and including the endpoint of 400 nm to 500 nm.

Most preferably the light emitted by the at least one treatment light source is monochromatic cw light of a wavelength as provided by any standard laser or LED known to a person skilled in the art, such as, but not limited to: 387 nm, 395 nm, 405 nm, 415 nm, 430 nm, 441.6 nm, 442 nm, 445 nm, 450 nm, 458 nm, 473 nm, 488 nm, 514 nm and/or 532. Using a standard semiconductor laser at 445 nm the wavelength of the laser may be varied between 442 and 448 nm by varying the temperature of the laser.

In the embodiment comprising the radiation of pulsed light the at least one treatment light beam originates from a laser system comprising at least one laser capable of emitting light with a pulse width in the range between seconds and femtoseconds. Generally, any pulse width ranging from cw light to femtoseconds, is of relevance to the present invention. Thus it is within the scope of the present invention that the emitted treatment light is pulsed with a width of between 1 second and 1 femtosecond and any pulse width falling into this interval is of relevance to the present invention. The pulse width may be in the long range such as in the second, millisecond or microsecond range, alternatively the pulse width may be in the nano-, pico- or femtosecond range. Examples of the two latter ranges are given herein below: In one embodiment the treatment light beam is pulsing with a pulse width shorter than substantially 60 microseconds, more preferably with a pulse width shorter than substantially 30 microseconds, with a pulse width shorter than substantially 10 microseconds, with a pulse width shorter than substantially 1 microsecond, with a pulse width shorter than substantially 500 nanoseconds, with a pulse width shorter than substantially 200 nanoseconds, with a pulse width shorter than substantially 100 nanoseconds, with a pulse width shorter than substantially 50 nanoseconds, with a pulse width shorter than substantially 5 nanoseconds, with a pulse width shorter than substantially 1 nanosecond, with a pulse width shorter than substantially 500 picoseconds, with a pulse width shorter than substantially 100 picoseconds. These ranges of pulse widths may be reached by direct modulation of e.g. a semiconductor laser, thereby keeping the cost and simplicity of the method and system according to the invention relatively low even when using a pulsed treatment light beam.

In another embodiment the treatment light beam is pulsing with a pulse width shorter than substantially 60 picoseconds, more preferably with a pulse width shorter than substantially 30 picoseconds, with a pulse width shorter than substantially 10 picoseconds, with a pulse width shorter than substantially 1 picosecond, with a pulse width shorter than substantially 500 femtoseconds, with a pulse width shorter than substantially 200 femtoseconds, with a pulse width shorter than substantially 100 femtoseconds, with a pulse width shorter than substantially 50 femtoseconds, with a pulse width shorter than substantially 5 femtoseconds.

The pulse energy follows the pulse width therefore in one embodiment the pulsing of the at least one treatment light beam preferably comprises pulsing with pulse energy in the area between joule and nanojoules. How this parameter relates to the pulse width is known to a person skilled in the art.

The pulse energy density of the treatment light beam preferably also depends on the two above parameters and thus the pulse energy density falls within the area as defined for the above parameters. Preferably, the pulse energy density is lower than substantially 1 Joule per square centimeter, lower than substantially 10 mJ/cm², lower than substantially 1 mJ/cm², lower than substantially 500 μJ/cm², lower than substantially 250 μJ/cm², lower than substantially 100 μJ/cm², lower than substantially 50 μJ/cm², lower than substantially 25 μJ/cm², lower than substantially 10 μJ/cm². More preferably, the energy of the pulsed light is below 1 μJ/cm², such as lower than 0.9 μJ/cm², such as lower than 0.8 μJ/cm², such as lower than 0.7 μJ/cm², such as lower than 0.6 μJ/cm², such as lower than 0.5 μJ/cm². Most preferably the energy of the pulsed light is below 0.40 μJ/cm², such as below 0.30 μJ/cm², such as lower than 0.20 μJ/cm².

Light Beams and Laser Beams Generally

For any type of treatment light beam, the at least one treatment light beam is preferably focused to a spot with a diameter of substantially 100 microns, more preferably 50 microns, 20 substantially microns, substantially 10 microns, substantially 5 microns, substantially 4 microns, substantially 3 microns, substantially 2 microns or substantially 1 micron.

The invention provides a method and a means for simultaneous use of multiple treatment beams entering the eye, each forming its own separate focus or target volume where a desired light-elicited chemical reaction or structural alteration change takes place, while avoiding side-effects that would follow from having the total energy delivered to the eye concentrated in a single focus. Thus, the intensity of the treatment light beam may be increased in the lens without increasing the intensity of the light imposed on the cornea, thereby protecting the cornea. The light thus emitted from the treatment light beams may be polarized so only the area of overlap receives enough energy to initiate the treatment (as in photomanipulating the lens). Multiple treatment beams may be provided by multiple light sources, by splitting a beam from a single light source or by splitting a beam of multiple multiplexed light sources.

Likewise, the invention provides a method and a system for simultaneous use of multiple probing beams entering the eye, each forming its own separate focus or target volume where a desired light-elicited response takes place, such a response being for instance fluorescence or another emission/radiation that enables control of target position, focus, and intensity in the lens.

Multiple light sources multiplexed into a single treatment beam may also be provided, e.g. by means of multiplexers, for example fiber based multiplexers. Multiplexing may be provided to increase the flux of the treatment beam, e.g. by multiplexing multiple substantially identical light sources, e.g. light sources of identical wavelength. Thereby the same power may be distributed over a larger area of the lens, thereby increasing the efficiency of the treatment and/or reducing the treatment time. However, multiplexing of multiple different light sources of different wavelengths, where each wavelength provides a specific response in the lens, thereby providing a co-operative effect between the multiple light sources. Multiplexing of multiple light sources may keep the cost down by using multiple low-cost sources instead of just one expensive light source.

In one embodiment of the invention the treatment light beam is combined with a secondary light source, e.g. by means of multiplexing, because it may also be that the at least one treatment control parameter is initiated and/or followed by a secondary source of radiation, such as a laser or LED. Thereby, the lens or selected part of same may e.g. be manipulated at one wavelength but probed for characterizing radiation/treatment control parameter using a different wavelength. And by multiplexing the treatment light beam with the secondary source of radiation it is substantially ensured that the treatment light beam and the probe beam is incident on the lens in the same selected part. This is especially important during scanning of the beam(s) relative to the lens. To avoid the treatment light beam and the probe beam incident on the lens in the same instant the beams may be pulsed and synchronized. One or more delay lines may ensure that pulses from the beams arrive at different instants.

Scanning of Beam Relative to Lens

Commonly it is not sufficient to photomanipulate the lens in one position only. Accordingly, in a preferred embodiment of the invention the focus of the light beam is scanned so as to treat at least one predefined volume, said volume being of a size enabling selective targeting of the lens substance and its sub-regions without damaging adjacent healthy or unhealthy tissue. Preferably the size of the said volume has a cross-section seen from the instrument that corresponds to the entire lens or specific parts thereof, or up to about 100 square millimetres, 10 square millimetres, 1 square millimetres, more preferably op to about 0.6 square millimetres, up to about 0.3 square millimetres, up to about 0.1 square millimetres, up to about 0.01 square millimetres, up to about 1000 square microns, up to about 100 square microns, up to about 10 square microns, up to about 1 square microns.

During treatment of the lens, the light beam may be scanned over at least a part of the lens. The scanning can be performed using various scanning patterns, such as meander scan, discontinuous line-by-line scan, a continuous line-by-line scan spiral scan, and/or circular scan. Furthermore, the scan velocity can be adjusted between scans or during a scan. The scan may furthermore be repeated one or several times. A preferred scan velocity is between 1 and 10.000.000 microns per second, more preferably between 10 and 500 microns per second, even more preferably between 50 and 250 microns per second, more preferably between 75 and 125 microns per second. However, in other embodiments the scan velocity may be between 1.000.000 and 10.000.000 microns per second, such as between 2.000.000 and 8.000.000 microns per second, such as between 3.000.000 and 6.000.000 microns per second, such as between 4.000.000 and 5.000.000 microns per second.

The whole lens may be treated by the treatment light beam. However, in practice it often suffers to treat the part of the lens that is viewable through the totally dilated pupil with normal incidence on the eye or maybe even just the central part of the lens (the nucleus). Thus, in some embodiments of the invention, the area of the lens that is treated concurrently by the treatment light beam is between 0.1 and 100 mm², such as between 0.1 and 1 mm², such as between 1 and 10 mm², such as between 10 and 20 mm², such as between 20 and 30 mm², such as between 30 and 40 mm², such as between 40 and 50 mm², such as between 50 and 60 mm², such as between 60 and 70 mm², such as between 70 and 80 mm², such as between 80 and 90 mm², such as between 90 and 100 mm². Preferably a substantially circular area.

In one embodiment of the invention the whole thickness of the lens is treated concurrently by the treatment light beam. A typical lens thickness is approx. 3-5 mm. Thus, in terms of the volume of the lens treated concurrently by the treatment light beam it may be a substantially cylinder shaped volume with a cylinder height of 3 to 5 mm, i.e. the thickness of the lens, and a cylinder cross sectional area of between 0.1 and 100 mm².

The means for scanning a beam over an object may comprise any of the means known to a person skilled in the art.

Retinal Protection

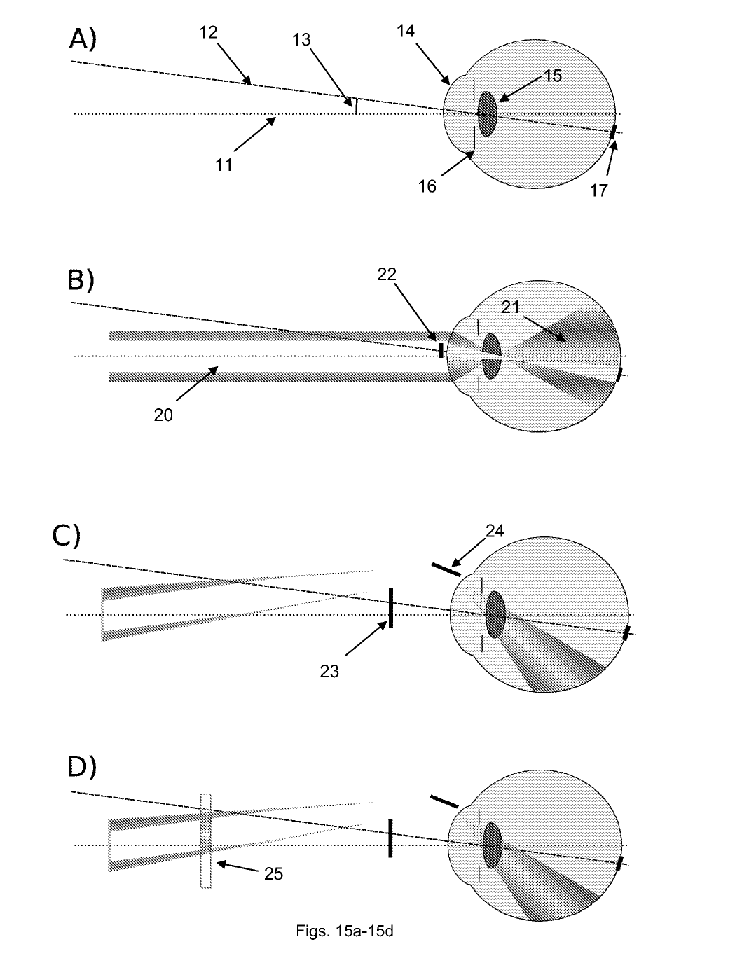

When exposing the eye to light there is a risk of damage, in particular retinal damage and particularly damage of the fovea. One embodiment of the invention provides means for radiation protection of the retina and/or the fovea. This may be provided by means of one or more beam blockers located in front of the eye in a substantially conjugate position to the fovea. Another solution could be to provide the treatment light beam at an indirect angle to the eye and direct the treatment light beam towards the lens by reflection means, such as a mirror. Different examples of fovea protection are illustrated in FIGS. 15A-D.

In a further embodiment of the invention retinal protection is provided by means of medical pre-treatment, e.g. by means of 13-cis-retinoic acid, that blocks the visual cycle.

Contact Lens on the Eye During Treatment

In the preferred embodiment of the invention a contact lens is mounted on the eye. The contact may be provided for immobilizing the eye during treatment (as described below). However, it may also be provided to keep the eye open, i.e. avoid blinks of the eye, and/or to keep the eye humid during treatment. The contact lens may also be provided to help focusing the treatment light beam into the lens. With at least a part of the focusing optics (i.e. inside the contact lens) being fixed to the eye a fixed focus distance to the eye is ensured independent of the position of the head of the individual to be treated, thereby increasing the safety of the treatment. One or more reflecting elements may also be incorporated in the contact lens thereby helping to guide the treatment light beams into the lens when multiple treatment light beams are applied.

Radiation Source

The source of radiation may be any source capable of emitting light at the desired wavelengths. Such sources include light sources based on thermal emission such as, but not limited to: candles, light bulbs, blackbody radiation, of which suitable filters are applied to block wavelengths not needed by the invention, lasers, LED's, discharge lamps and/or sunlight.

Thus the light source may be a discharge lamp, such as a Xenon, Krypton, or Deuterium discharge lamp, or even sunlight, to which suitable filters are applied to block wavelengths not needed by the invention.

Preferably the light source is a laser source based on a gaseous or metal-vapor medium, such as, but not limited to: HeCd, HeNe, Ar-ion, Nitrogen or Krypton, emitting a narrow bandwidth light emission in the wavelength range needed by the invention.

More preferably the light source is a laser based on a solid state gain medium, like, but not limited to, Nd:YAG, Nd:YLF, Alexandrite, Ti:Sapphire, Ruby, Cr:LiSAF, Cr:Forsterite. However, the light source may be based on one or more gas lasers. The laser source is used either in its basic mode of operation or by using nonlinear optics are wavelength converted into a wavelength better suited for the invention. Examples hereof include, but are not limited to: Frequency doubled Nd:YAG emitting radiation at 532 nm, 473 nm or 660 nm, Frequency doubled Cr:LiSAF emitting laser radiation in the range 390 nm to 530 nm, frequency doubled Ti:Sapphire emitting laser radiation around 400 nm.

Most preferably the light source used will be based on semiconductor lasers, also termed laser diodes, emitting radiation in the wavelength range usable by the invention. Examples of wavelengths include, but are not limited to: 375 nm, 395 nm, 405 nm, 441.6 nm, 442 nm, 445 nm, 450 nm, 470 nm, 488 nm. The emitted radiation can also be wavelengths converted into wavelengths more suitable for the invention.

Thus, the light source preferably comprises a compact laser source delivering continuous wave and/or short, tunable pulses of laser light with an adjustable delay of duration between consecutive pulses.

Preferably, the light is emitted by continuous wave lasers such as, but not limited to: A 385 nm laser, 405 nm laser, a 420 nm laser, a 441.6 nm laser, a 442 nm laser, a 445 nm laser, a 450 nm laser, a 458 nm laser, a 488 nm laser, a 514 nm laser, or a 532 nm laser.

Preferred embodiments include, but are not limited to: a cw laser at 405 nm generated by frequency doubling of an 810 nm tapered diode laser; an external phase-locked doubler resonator used to generate 300 mW of light; a cw 532 nm laser with a cw diode-pumped solid state second harmonic Nd:YAG laser.

The polarization of the radiation source may if applicable as known to a person skilled in the art be controlled by using polarizers, and waveplates, in order optimize the treatment parameters. The optimal polarizations include, but are not limited to: linear, circular, elliptical, unpolarized. The optimal polarization may be dependent on wavelength, light intensity, pulse length, and direction of propagation relative to treatment area.

In yet another preferred embodiment, the treatment laser system comprises a photonic crystal fiber, where said fiber is preferably pumped by a diode laser.

It is of relevance to note, that the system herein disclosed and the method to be performed herewith do not involve corneal treatment. The methods employed during corneal treatment typically involve a destructive step of cutting a flap in the eye using laser light of 1000 nm or more and abrading the cornea with light of 200 nm or less. Light above app. 800 nm may be damaging to the eye and light below 260 nm does not reach the lens. Other methods are invasive unlike the present invention, include the use of dye in the cornea, or comprise a step of destroying tissue within the lens and/or lens capsule all of which are aspects that are of no issue to the present invention.

Immobilization

The human eye exhibits micro-movements with a frequency in the order 10 Hz. These micro-movements are involuntary and it is therefore not possible for a subject receiving photomanipulation to suppress these movements by will. In one preferred embodiment of the present invention spatially accurate photomanipulation is obtained by mechanically immobilizing the living eye, wholly or partly, during treatment, by mechanical contact with the surface of the eye or by mechanical contact to a contact lens mounted on the eye. Furthermore, this mechanical immobilization preferably further comprises a fluid interface in the said mechanical contact, as well known in the art, and/or an application of suction to reinforce the said mechanical contact also well known in the art.

Tracking of eye movements and/or orientation is an alternative or supplement to immobilization of the eye rendering a high potential for accuracy. Specifically if coupled to a response system which provides real-time beam adjustment, so that eye movements are nullified relative to the system.

Accordingly, a preferred embodiment of the invention comprises a method of tracking the movement of the eye by imaging of the eye on at least one light detector. Preferably the said at least one of the said light detector(s) comprises a camera and preferably the movement of the eye is found by tracking reference points in the eye. To obtain even higher accuracy the said light detectors may be spatially separated so a 3D perspective may be calculated. Accordingly, it is preferred that each light detector views the eye from different angles.

In another preferred embodiment of eye tracking according to the invention, the orientation of an eye in space is monitored by simultaneously monitoring the surface or anterior part of the eye and the fundus (posterior inside of the eye) and calculating the orientation of the eye in space.

The system in the present invention may be combined with devices that are capable of performing the above described actions and/or measurements for immobilization of the eye/lens. Such devices are known to persons skilled in the art.

System Features

Thus, the present invention relates to a system for prevention, treatment and/or amelioration of a disease and/or disorder which is either related to a lens of an eye and/or which may benefit from the treatment of said lens, said system comprising:
a) focusing means for focusing a treatment light beam of a wavelength substantially between 320 nm and 800 nm into a selected part of the lens and/or its constituents collectively or selectively where treatment is intended to occur, and
b) means for emitting and/or unblocking said treatment light beam, thereby inducing changes in the lens and/or its constituents, and
c1) means for stopping and/or blocking said treatment light beam after a predetermined period of time, and/or
d1) means for measuring one or more types of radiation from said selected part, and
d2) means for processing said one or more type of radiation from said selected part, and
d3) means for adjusting, based on at least part of the output of the means for processing, at least one of the parameters for said treatment light beam: focus, intensity, wavelength, pulse length, repetition frequency, and pulse train length of said treatment light beam.

Thereby optimally photomanipulating said lens and/or its constituents thus treating, preventing and/or ameliorating said disease or disorder.

Also, the present invention includes the means to perform the method disclosed in the below of prevention, treatment and/or amelioration of a disease and/or disorder which is either related to a lens of an eye and/or which may benefit from the treatment of said lens, the method comprising the exposure of said lens to radiation of short wavelength substantially between 320 nm and 800 nm, thereby inducing changes in the lens and/or its constituents, comprising:
a) means for focusing a treatment laser beam into a selected part of the lens and/or its constituents collectively or selectively where treatment is intended to occur;
b) means for pulsing of said treatment laser beam;
c) means for scanning the treatment laser beam over the lens or at least parts thereof with a constant or varying velocity;
d) means for measuring one or more types of radiation from the said selected part and means for utilizing this measurement to decide to stop the said treatment laser beam or to adjust at least one of the parameters: focus, intensity, wavelength, polarization, pulse length, repetition frequency, pulse train length, scan velocity, size of scanned volume, scan repetitions, and scan pattern of said treatment laser beam;

Treatment

The present invention provides the means and methods for the prevention, treatment and/or amelioration of diseases and/or disorders which are either related to the eye lens of an eye and/or which may benefit from the treatment of the lens. The method comprises the exposure of a lens in need of treatment to radiation, thereby inducing changes in said lens and/or its constituents.

The processes that occur during radiation with light are also known as photolysis, photodissociation, or photodecomposition and are chemical reactions in which a chemical compound is broken down by photons. The terms are used interchangeably herein together with photobleaching and photomanipulation. Photolysis is not limited to visible light, but must have enough energy to break up a molecule. The photon is likely to be an electromagnetic wave with the energy of visible light or higher, herein such as visible and ultraviolet light. The direct process is defined as the interaction of one or more photons with one or more target molecules.

It is an object of the present invention to reduce the coloration of a lens by photolysing/photobleaching the lens with visible or UV light. It is a further object of the present invention to increase the accommodation ability of a lens by photomanipulating the lens with visible or UV light, thus reducing the stiffness of the lens.

The exact photochemical processes involved in the bleaching process are unknown. The age-related changes in the human lens are partly caused by formation of advanced glycation end products, which have been reported to produce active oxygen species upon exposure to UVA (Masaki et al., 1999). Hypothetically, reactive oxygen species produced by laser irradiation could lead to photochemical reactions that cleave the chemical bonds in the lens which have formed during age-related denaturation of lens proteins, thus leading to restoration of the properties of the younger lens.

Method

The present application thus provides a method of prevention, treatment and/or amelioration of a disease and/or disorder which is either related to a lens of an eye and/or which may benefit from the treatment of said lens, the method comprising the exposure of said lens to radiation of short wavelength substantially between 320 nm and 800 nm, thereby inducing changes in the lens and/or its constituents, comprising:
a) focusing a treatment light beam into a selected part of the lens and/or its constituents where treatment is intended to occur;
b) emitting light from said treatment light beam;
c) measuring one or more types of radiation from the said selected part and utilizing this measurement to decide to stop the treatment light beam or to adjust at least one of the parameters: focus, intensity, wavelength, pulse length, repetition frequency, and pulse train length of said treatment light beam,

whereby the photomanipulation is effectively monitored allowing for an accurate and optimum application of photo energy.

A second embodiment of the invention regards a method of prevention, treatment and/or amelioration of a disease and/or disorder which is either related to a lens of an eye and/or which may benefit from the treatment of said lens, the method comprising the exposure of said lens to radiation of a wavelength substantially between 320 nm and 800 nm, thereby inducing changes in the lens and/or its constituents, comprising:
a) focusing a treatment light beam into a selected part of the lens and/or its constituents collectively or selectively where treatment is intended to occur;
b) emitting light from said treatment light beam;
c) stopping said treatment light beam after a predetermined period of time,

whereby the photomanipulation is provided in a certain period of time, thereby avoiding the monitoring part.

Furthermore there is provided a method of prevention, treatment and/or amelioration of a disease and/or disorder which is either related to a lens of an eye and/or which may benefit from the treatment of said lens, the method comprising the exposure of said lens to radiation of short wavelength substantially between 320 nm and 800 nm, thereby inducing changes in the lens and/or its constituents, comprising:
a) focusing a treatment light beam into a selected part of the lens and/or its constituents collectively or selectively where treatment is intended to occur;
b) emitting light from said treatment light beam;
c) measuring one or more types of radiation from the said selected part and utilizing this measurement to decide to stop the said treatment light beam or to adjust at least one of the parameters: focus, intensity, wavelength, pulse length, repetition frequency, and pulse train length of said treatment light beam.

thereby photomanipulating the lens and/or its constituents thus treating, preventing and/or ameliorating said disease or disorder.

Furthermore there is provided a method of prevention, treatment and/or amelioration of a disease and/or disorder which is either related to a lens of an eye and/or which may benefit from the treatment of said lens, the method comprising the exposure of said lens to radiation of short wavelength substantially between 320 nm and 800 nm, thereby inducing changes in the lens and/or its constituents, comprising:
a) focusing a treatment laser beam into a selected part of the lens and/or its constituents collectively or selectively where treatment is intended to occur;
b) emitting light from said treatment laser beam;
c) stopping said treatment light beam after a predetermined period of time,

thereby photomanipulating the lens and/or its constituents thus treating, preventing and/or ameliorating said disease or disorder.

Another embodiment relates to a method for non-invasive prevention, treatment and/or amelioration of a disease and/or disorder which is either related to a lens of an eye and/or which may benefit from the treatment of said lens, the method comprising the exposure of said lens to radiation of short wavelength substantially between 320 nm and 800 nm, thereby inducing changes in the lens and/or its constituents, comprising:
a) focusing a treatment light beam into a selected part of the lens and/or its constituents collectively or selectively where treatment is intended to occur;
b) emitting light said treatment light beam;
c) scanning the treatment light beam over the lens or at least parts thereof with a constant or varying velocity;
d) measuring one or more types of radiation from the said selected part and utilizing this measurement to decide to stop the said treatment light beam or to adjust at least one of the parameters: focus, intensity, wavelength, polarization, pulse length, repetition frequency, pulse train length, scan velocity, size of scanned volume, scan repetitions, and scan pattern of said treatment light beam.

Another embodiment relates to a method for non-invasive prevention, treatment and/or amelioration of a disease and/or disorder which is either related to a lens of an eye and/or which may benefit from the treatment of said lens, the method comprising the exposure of said lens to radiation of short wavelength substantially between 320 nm and 800 nm, thereby inducing changes in the lens and/or its constituents, comprising:
a) focusing a treatment light beam into a selected part of the lens and/or its constituents collectively or selectively where treatment is intended to occur;
b) emitting light said treatment light beam;
c) scanning the treatment light beam over the lens or at least parts thereof with a constant or varying velocity;
d) stopping said treatment light beam after a predetermined period of time or when a predetermined part of the lens has been covered by the scan.

A further embodiment regards a method of providing optimum benefit of treatment while minimizing the risk of damage consisting of controlling the patient's body fluid levels, e.g. serum or plasma values, of one or more vitamins, or other physiological or extrinsic substances with the aim of excluding patients with values for riboflavin or other photosensitizing agents above a defined threshold or excluding patients with insufficient levels of substances that may provide protection against adverse effects.

Also provided is a method of providing optimum benefit of treatment while minimizing the risk of damage consisting of applying exposure to light of duration 0.000001 second to 10000 seconds with a spectral composition ranging from white light to one or more selected spectral bands of visible light, including such band or bands that most optimally fit the aborption peak or peaks of one or more retinal photopigments, including the photopigments of the rod and the cone photoreceptors and the photopigments of the retinal ganglion cells or any combination of the photopigments of the rods, cones, ganglion cells, and other light-sensitive cells of the eye, the luminance of the said flash of light ranging from 10 candela per square meter to 1 million candela per square meter and the area of exposure including all or one or more parts of the retina, the desired effect of the light exposure comprising the bleaching of retinal photopigments such that subsequent exposure of the eye to radiation targeting conditions in the lens can be made under conditions that reduce the risk of exposing the retina to harmful types or levels of radiation.

A specific embodiment hereof includes flash illumination of the eye using light diffusely illuminating and being reflected from a large reflecting surface placed in front of a human subject with open eyelids, the light consisting of the wavelenghts 480 nm, 500 nm, and 550 nm or any combination thereof and any extension of the bandwidths of the said spectral lines up to and including a continuous band ranging from 450 nm to 600 nm.

A method of providing optimum benefit of treatment while minimizing the risk of damage comprising the repeated exposure of the eye to light of the said characteristics, with optional intraprocedural monitoring of light sensitivity, including the provision of visual objectives that alert the patient to respond in a manner, such as activating an electric contact, indicating that a renewed bleaching exposure is needed and prompting an operator or a machine to effect renewed bleaching.

An additional embodiment regards a method of providing optimum benefit of treatment while minimizing the risk of damage consisting of inhibiting processes in the retina that occur in response to illumination, including but not limited to those processes that are responsible for vision and for controlling the diurnal cycle.

Individual to be Treated

The lens, and thus the eye of an individual to be treated may be any animal or human being. The method is generally practiced on a living eye of an animal or a human being. Preferably the individual to be treated is a mammal, such as but not limited to a dog, cat, primate, horse, cow or human being.

Most preferably the herein disclosed method is used for the treatment of a lens of a human being. The treated elements being any of the lens, its capsule and/or its constituents.

Medication

To make the lens more susceptible to photomanipulation and/or more susceptible to remove bi-products of the photomanipulation, it is for some applications advantageous to condition the eye prior, during, or post photomanipulation or assessment. Accordingly, in a preferred embodiment the invention comprises a method for conditioning the eye prior to treatment, during treatment, post treatment, prior to assessment, during assessment or post assessment by applying at least one of the following: heat, cold, magnetic field and/or a pharmaceutical. The pharmaceutical may be a compound capable of sedating and/or anaesthetizing the eye and/or the surround tissue.

Similarly, in another preferred embodiment the invention comprises the administration of adjuvant pharmaceuticals. Preferably, these pharmaceuticals quench free radicals in the eye. Such free radicals may arise as an undesired bi-product of the photomanipulation and are preferably disposed by means other than interaction with healthy tissue. Furthermore the addition prior to, during or after treatment of a biologically acceptable compound capable of breaking chemical bonds between and/or within the lens proteins, f. ex. breaking the disulphide bindings between the lens proteins, is also an aspect of the present invention. Examples of such compounds include, but are not limited to glutathione, thiols and their derivatives.

A method of providing optimum benefit of treatment while minimizing the risk of damage consisting of inhibiting the regeneration of visual pigment in the retina by pharmacologically blocking enzymes, receptors, channels, genes or cells that contribute to the continuous restoration of light sensitivity in the living eye, including but not limited to methods comprising the administration of pharmaceutical agents such as fenretinide, 13-cis-retinoic acid (isotretinoin), 11-cis-retinol, 11-cis-retinal, 11-cis-retinyl bromoacetate, acitretin, etretinate, 4-oxo-isotretinoin, motretinide, retinaldehyde, all-trans-retinyl bromoacetate, all-trans-retinyl chloroacetate, and retinoyl betaglucoronide.

### DETAILED DESCRIPTION OF THE DRAWINGS

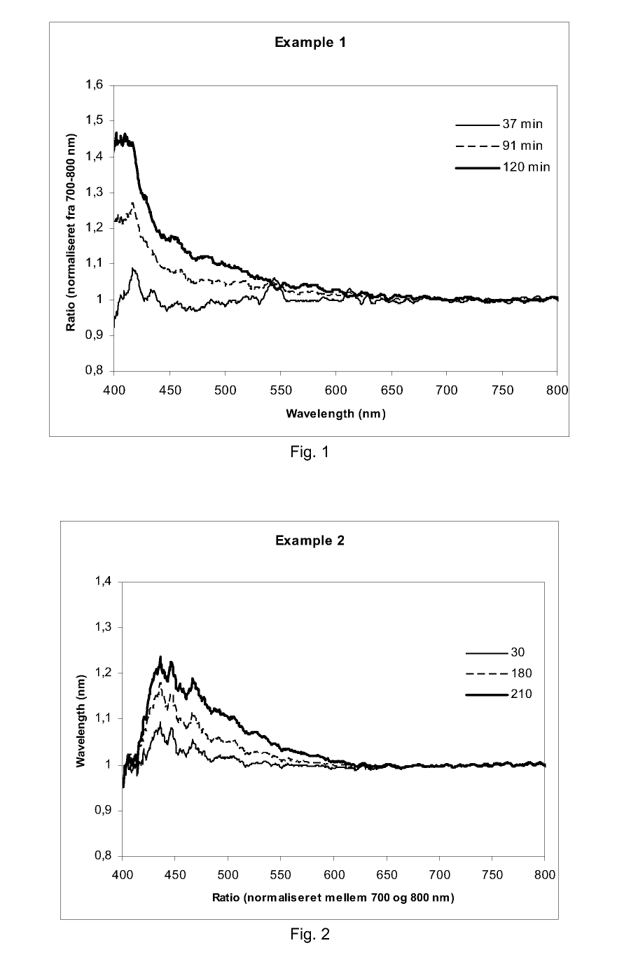

FIG. 1: Transmission spectra pre and post treatment of young lens, see ex. 1.

FIG. 2: Transmission spectra pre- and post treatment of old lens, see ex. 2.

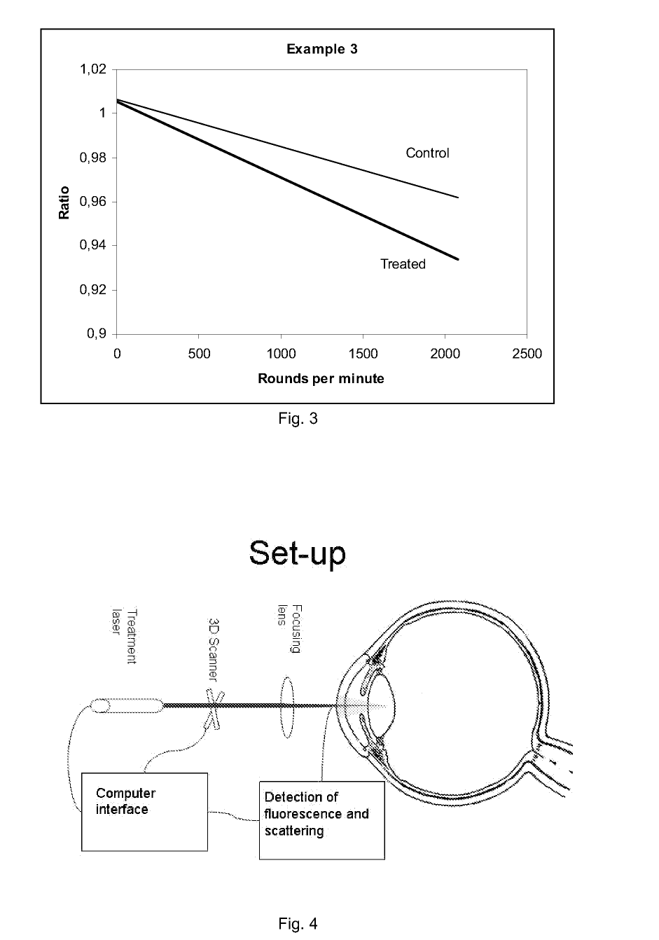

FIG. 3: Elasticity measures of treated vs. non-treated lenses, see ex. 3.

FIG. 4: Schematic drawing of the treatment system. The treatment system includes a treatment laser beam which is scanned in all directions. The treatment laser beam is focused to a predefined area inside the lens by a focusing lens. The treatment is monitored and predetermined by use of intrinsic optical parameters of the lens such as lens auto-fluorescence and light scattering. The treatment is controlled by a computer interface which controls the treatment parameters (such as laser intensity and dosimetry and scanning pattern) and adapts them according to feed-back from the monitoring parameters.

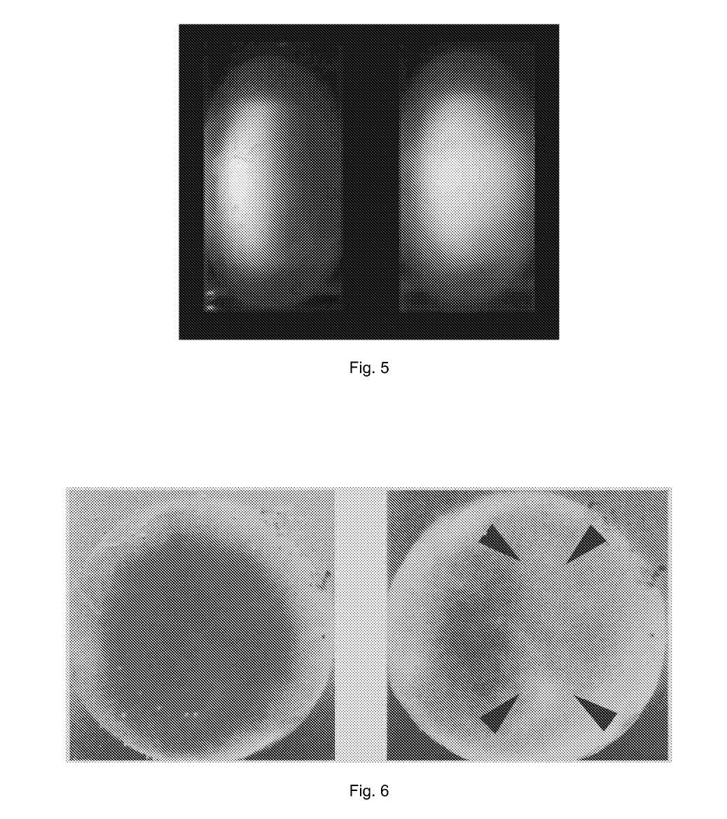

FIG. 5: Gray-scale photograph showing auto-fluorescence emitted from a lens (side-view) at the start of exposure to a 405 nm continuous wave laser (left) and at the end of laser exposure (right). Photographs were taken perpendicularly to the direction of the laser beam.

FIG. 6: Photographs showing the same lens before exposure to a 405 nm continuous wave laser (left) and after exposure (middle). Arrowheads on the middle photograph marks the boundaries of the laser beam.

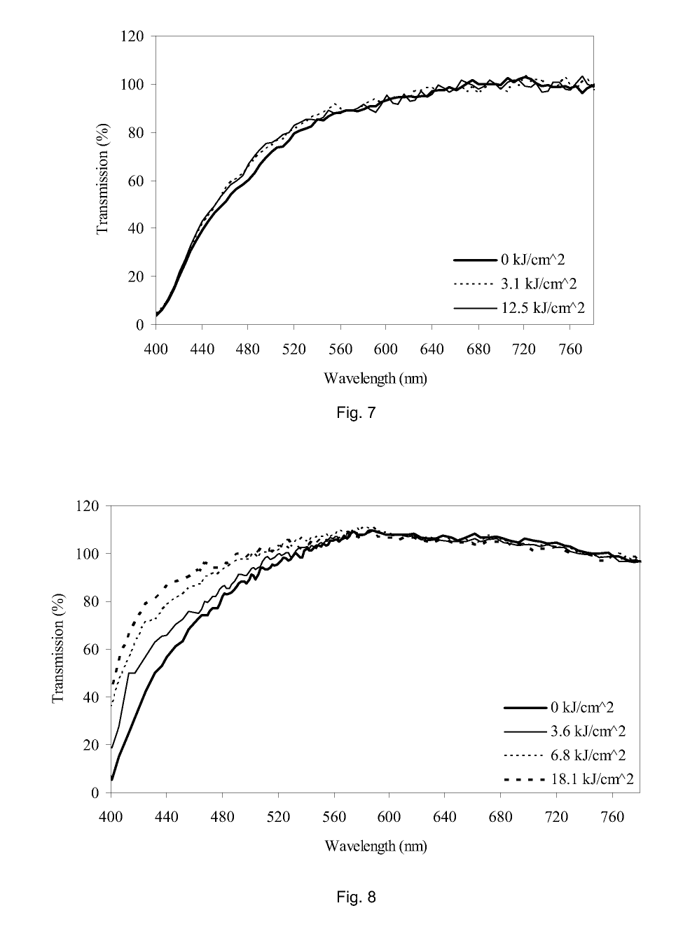

FIG. 7: Transmission spectra of the same donor lens (aged 56 years) before and after exposure to increasing doses of irradiation from a 405 nm continuous wave laser.

FIG. 8: Transmission spectra of the same human donor lens (aged 72 years) before and after increasing doses of irradiance from a 532 nm continuous wave laser.

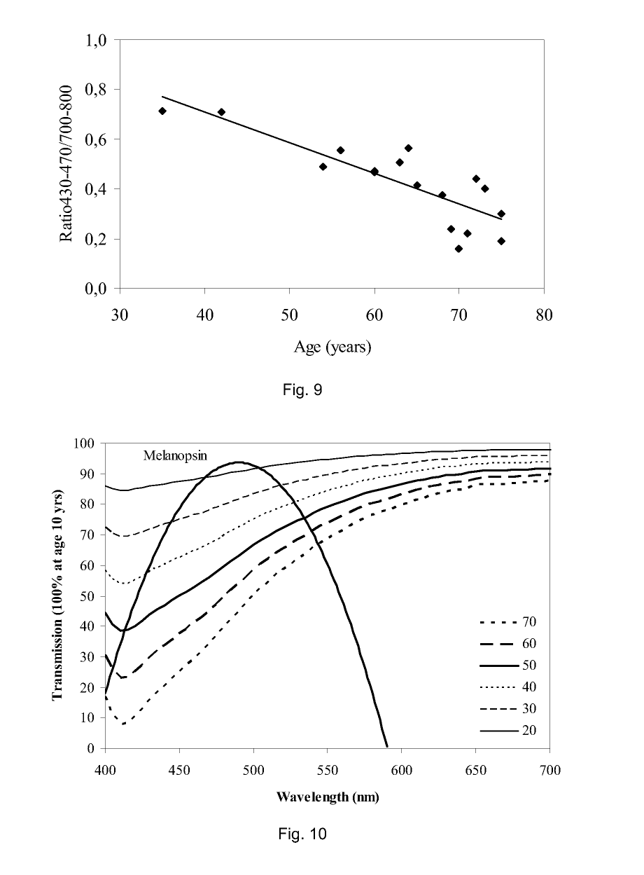

FIG. 9: The figure shows the age-related decrease in the transmission of light from 430 to 470 nm relative to the transmission of light from 700 to 800 nm. The curve is based on equation 1 (see example 4 for details).

FIG. 10: Model curves of transmission in the human lens at different ages (20 to 70 years of age) relative to the transmission of a 10 year old lens. The absorption curve of melanopsin is also shown (redrawn from Hankins et al) to illustrate the predilection for age-related transmission loss to affect the blue end of the visible spectrum where the melanopsin photopigment is stimulated.

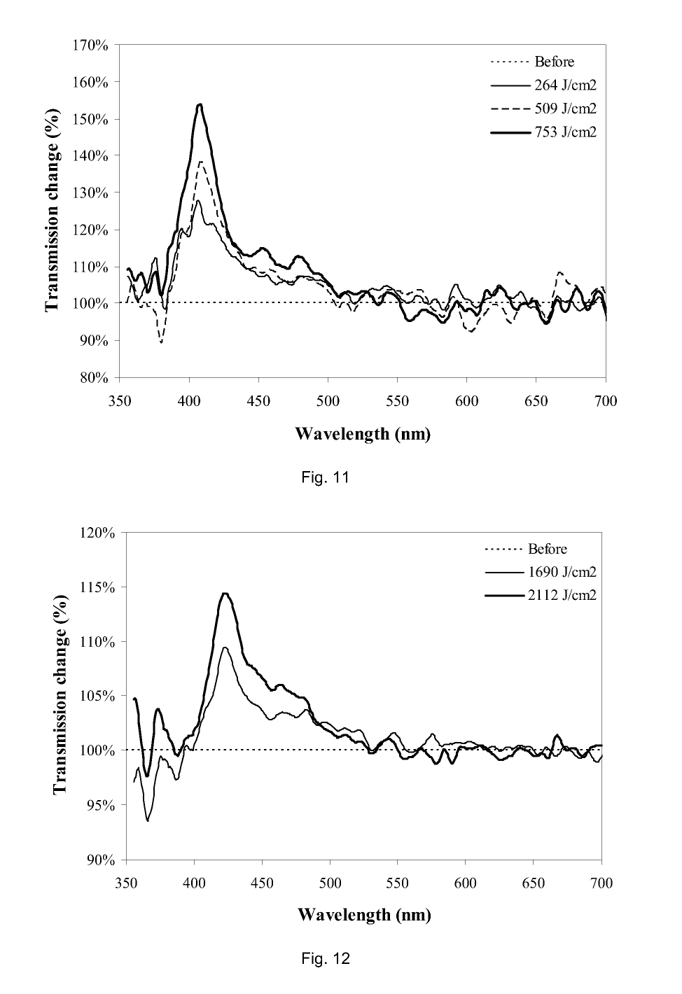

FIG. 11: Changes in transmission in the human lens after irradiation with a 355 nm pulsed laser. The transmission before irradiation was set at 100% for all wavelengths.

FIG. 12: Changes in transmission in the human lens after irradiation with a 400 nm femtosecond pulsed laser. The transmission before irradiation was set at 100% for all wavelengths.

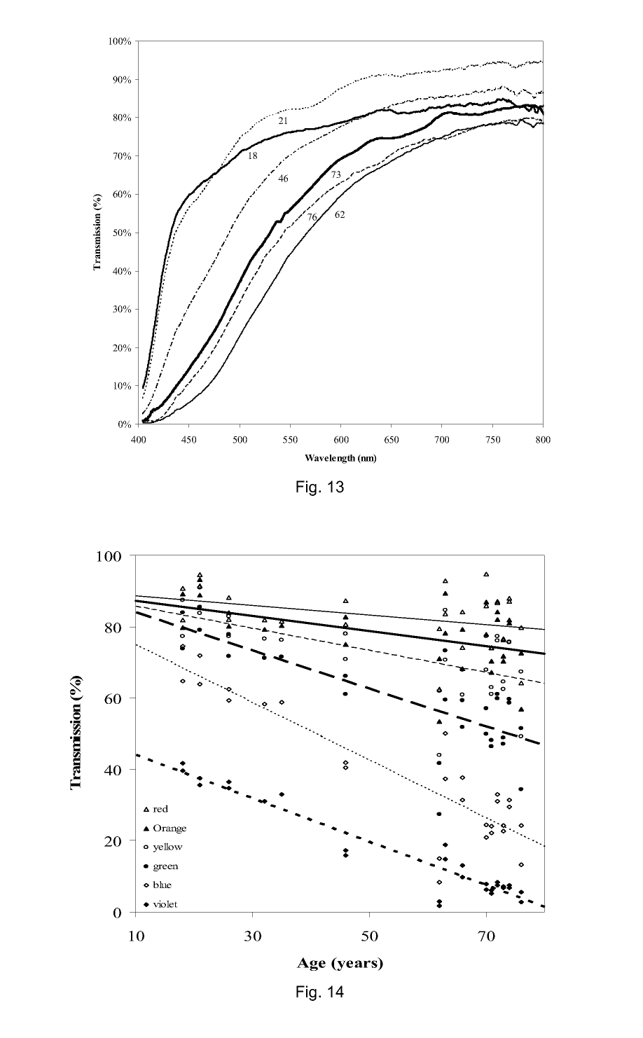

FIG. 13: Spectral transmission in vitro of human donor aged 18, 21, 46, 62, 73, and 76 years of age (same lenses as depicted in FIG. 12).

FIG. 14: Lens transmission in vitro in selected spectral bands for 28 human lenses with regression curves (see Table 1) describing the average reduction in transmission with age. The markers indicate the following spectral bands: violet (400-449 nm), blue (450-489 nm), green (490-559 nm), yellow (560-589 nm), orange (590-629 nm), red (630-699 nm), and infra-red (700-800 nm) appearing in that order in the figure, counted from the bottom.

FIGS. 15A-D are schematic illustrations of different exemplary treatment setups, in particular setups to protect the fovea.

FIG. 15A is a schematic cross section of the eye. The sectioning is provided as it would appear to an observer looking down on a patients head. The eye shown is the thus the right eye. The small angle 13 between the Optical Axis 11 and the Visual Axis 12 is called angle-alpha, and is usually of about five degrees, but can vary from −2 to fifteen degrees. The lens 15 is located right behind the iris 16 and the cornea 14. The small spot 17 on the retina is the area of acute visual sensitivity in the eye, containing the color cones.

FIG. 15B illustrates a wide collimated beam 20 with normal incidence on the eye. A protective stop 22 has been placed in the eye, at the conjugate position of the cornea-lens system in order to be imaged at the retina, as a shadow region to protect the fovea during treatment. The protective stop 22 can be moved relative to the optical axis to account for varying angle-alpha and fovea locations.

FIG. 15C illustrates off-axis incidence of the treatment beam or beams. The off axis incidence allows for the beam to treat the lens without ultimately to cover the fovea region of the retina, markedly reducing the risk of the treatment. A protective stop 23 has been incorporated for safety and to protect the iris from the treatment beam. The angle of the treatment beam depends on lens parameters such as age, thickness, diameter, index profile, anterior curvature and posterior curvature. This setup can either be used in reflection mode (as shown with reflective element 24) or in direct mode, wherein the treatment beam is provided at an angle to the optical axis. Several beams can be used simultaneously, e.g. by treating from several angles or using several mirrors that will direct the treatment beam into the eye at different angles to the optical axis. A multi-mirror setup could consist of an ophthalmic gonio lens with 4 mirrors in contact mode, thereby multiplexing 4 beams simultaneously.

FIG. 15D illustrates how to provide an increased control of the treatment beam by means of a spatial light modulator, either in intensity modulation mode, phase modulation mode or a combination thereof. Other modulation sources could be of electro-optic modulators, acusto-optic modulators, piezo-controlled mirrors or lenses, diffractive elements or holograms and adaptive optics systems. Light modulation allows a change of the beam profile and local intensity of the treatment beam, allowing for treatment of inhomogeneous diseases. A large change in the phase-front will result in a very small depth of field thereby increasing the treatment accuracy. The control of the light modulator is obtained from the diagnostic system in feedback mode.

### EXAMPLES

Methods

Biological Material

Human donor lenses were procured within 24 hours post mortem. The lenses were kept in minimal essential medium under cool conditions until they could be used for the experiments. For the experiments, the lens capsules were gently removed using forceps and the lens was placed between two glass mounting plates which were kept apart by an adjustable spacing system to allow for individual differences in lens size.

Laser Systems

Four different laser systems were examined: a pulsed nanosecond laser at 355 nm, a pulsed femtosecond laser at 400 nm, a continuous wave (cw) laser at 405 nm, and a cw laser at 532 nm. Laser energy output was measured using a thermopile detector and could be adjusted using a graded neutral density filter.

Pulsed 400 nm Laser

A femtosecond Ti:Sapphire laser centered at 800 nm was frequency doubled to 400 nm using a Coherent OPA 9400 optical parametric amplifier. The laser system consisted of a mode locked Ti:Sapphire laser (Mira 900, Coherent) and a regenerative amplifier (RegA, Coherent). Pulse duration was characterized using an autocorrelator. It was in the range of 150-250 fs (10⁻¹⁵ seconds) at 400 nm. Repetition rate was set to 275 kHz. The system delivered pulses with a pulse energy of 2 μJ but pulse energy was adjusted to the desired level using a graded neutral density attenuation filter.

Continuous Wave (CW) 405 nm Laser

The cw laser at 405 nm was the second harmonic of an 810 nm tapered diode laser. An external phase-locked doubler resonator was used to generate 150 mW of light but the power was lowered in the experiments.

Continuous Wave 532 nm Laser

The 532 nm laser was a cw diode-pumped solid state second harmonic Nd:YAG laser (LSR532U-200, Lasever, China).

Transmission Spectra

Transmission spectra were recorded during the experiments using an Avantes Spectrometer (AvaSpec-2048-2, Avantes BV, The Netherlands) as the transmission of a white light source through the laser exposed area of the lens. To ensure that transmission spectra were obtained at the exact location of the laser exposure, the area of interest was defined by fixating a circular aperture of 1.4 mm in diameter on the mounting plates. The laser beam cross section was kept greater than the aperture ensuring that the entire area of the aperture was exposed. Transmission spectra were normalized to a nominal transmission of 100% between 700 and 800 nm.

### Example 1

### Increase in Transmission, Young Lens

A 51 year old human donor lens was treated with a 405 nm diode cw laser with a power of approximately 40 mW/cm² for 2 hours, corresponding to a total dose of approximately 280 J/cm². Transmission spectra were recorded during exposure and showed a 40% increase in transmission compared to the transmission before exposure began (see FIG. 1). The increase in transmission is greatest in the blue-green end of the spectrum.

### Example 2

### Increase in Transmission, Old Lens

A 74 year old human donor lens was exposed to the same amounts of radiation as in example 1. The increase in transmission was less than in example 1 but the spectral range of the bleaching was much broader (see FIG. 2). This reflects that the lens in example 2 was much older and had a much more densely, colored lens.

### Example 3

### Increased Elasticity

A human donor lens was exposed to approximately 300 mW/cm² of 405 nm diode cw laser for 5 hours. FIG. 3 shows the response to centrifugal forces as a ratio of the height of the lens before rotation and with increasing speed of rotation (shown on the x-axis in rounds per minute). The lens of the right eye was kept as a control lens and the left lens from the same donor was treated.

### Example 4

Lens Transmission

Light transmission of human donor lenses showed marked changes with age. Primarily the transmission of light in the blue-green range of the spectrum was affected (FIG. 9 and Eq. 1). There was some inter-individual variation in transmission spectra whereas the transmission spectra of the right and left eye from the same donor were nearly identical. To quantify the spectral response of the individual lenses, we defined a ratio, Ratio_{430-470/800-800}, of the average transmission in the range 430 nm to 470 nm relative to the average transmission in the 700-800 nm range. This ratio is plotted for the lenses used in the experiments in FIG. 9, which shows a clear age-dependent relation. A linear fit to the data can be used to express this relation, see Equation 1 (Eq 1) where A is age:

Ratio_{430-470/700-800}=1.20320−0.01235␣A (p<0.0001) Eq. 1

Laser Exposure

Exposure to laser light at 400 nm (a femtosecond pulsed laser) and 405 nm (continuous wave) laser resulted in a macroscopically significant loss of the yellow age-related coloration of the human donor lenses corresponding to an increase in transmission of light from 400 to 550 nm (FIGS. 6 and 7). The photobleaching did not show any sign of regression for an observational period of 2 weeks. The bleaching effect showed a dose-response relationship with the amount of irradiation. The increase in transmission upon exposure to 405 nm monochromatic laser light corresponded to the transmission of lenses which were 15 to 20 years younger according to Eq. 1. Very similar results were produced no matter whether a pulsed or a cw laser system was used.

FIG. 5 is a gray-scale photograph of the autofluorescence in a lens at the beginning and at the end of exposure to the 405 nm cw laser. Initially, the fluorescence is clearly restricted to the anterior part of the nucleus but as exposure continues and the blue-absorbing compounds in the lens are photobleached, the autofluorescence is emitted from the entire nucleus.

Exposure to a 532 nm cw laser lead to a minor increase in transmission from 440 to 540 nm (FIG. 8). The maximum effect was obtained at a radiance of 3.1 kJ/cm² and no further effects were noted even after doses up to 12 kJ/cm².

Conclusion

This study demonstrated that significant bleaching of the yellow chromophores of the human lens can be obtained using 400 or 405 nm pulsed or continuous wave laser radiation corresponding to the lens of a one to two decade younger donor and without inducing visible focal lesions in the lens. Likewise bleaching effects were produced at 532 nm.

### Example 5

The optical performance of the lens of the eye deteriorates with age, reducing the intensity of the light reaching the retina and degrading the image quality. While some of these changes impair visual function, others may be beneficiary to the eye. Notably, the gradual yellowing of the lens may protect the aged retina from phototoxic damage produced by short wavelength light. In this example the age-related changes in transmission of light by non-cataractous human lenses is characterized by measuring the transmission of white light in vitro in 28 intact lenses from 15 different human donors aged 18 to 76 years of age.

Measurement

The transmission of visible and near-infra red light (400-800 nm) was studied with the intact lenses placed in 5 mm pathlength quartz cuvettes (Starna Scientific Ltd, Hainault, UK) filled with a neutral saline solution containing (in g/l) 8.00 NaCl, 0.40 KCl, 0.10 Na₂HPO₄, 1.00 glucose, 2.38 Hepes buffered with NaOH to a pH of 7.4. The transmission was calculated as the ratio of the intensity of white light transmitted by a human lens compared to the transmission of light by a blank quartz cuvette filled with the saline solution after subtraction of a background level of light:

$$Transmission = \frac{{Spectrum}_{lens} - {Spectrum}_{background}}{{Spectrum}_{cuvette} - {Spectrum}_{background}}$$

The transmission was measured through the axial portion of the lens with the anterior side facing the light source. A supercontinuum white light source was used producing a collimated beam of light from 395 nm to 2100 nm with a beam diameter of approximately 1 mm. After passing through the cuvette containing the human lens, the light was collected by an integrating sphere coupled to a spectrometer by an optical fibre and controlled by a computer programme.

Results

Older lenses were larger and more densely yellow than younger lenses but with individual variations that were related to systemic health factors such as diabetes mellitus. The transmission spectra demonstrated a monotonous decrease in transmission from the red to the blue end of the spectrum with age. This is shown in FIG. 13 showing the spectral transmission vs. wavelength.

TABLE 1

| Colour | Slope (a) | Intercept (b) | p-value | R²-value |
|---|---|---|---|---|
| Violet (400-449 nm) | 0.6 | 50.1 | <0.0001 | 0.92 |
| Blue (450-489 nm) | 0.8 | 82.9 | <0.0001 | 0.81 |
| Green (490-559 nm) | 0.5 | 88.3 | <0.0001 | 0.61 |
| Yellow (560-589 nm) | 0.3 | 88.8 | 0.0007 | 0.37 |
| Orange (590-629 nm) | 0.2 | 89.4 | 0.0092 | 0.23 |
| Red (630-699 nm) | 0.1 | 90.0 | 0.064 | 0.13 |
| Near infrared | 0.1 | 92.6 | 0.0147 | 0.24 |
| (700-800 nm) | | | | |

The transmission was modelled as a function of age for each of the seven different spectral bands that are attributed by colour names (Table 1 and FIG. 14). FIG. 14 shows Lens transmission in the selected spectral bands for the 28 human lenses with regression curves (see Table 1) describing the average reduction in transmission with age. The markers indicate the following spectral bands: violet (400-449 nm), blue (450-489 nm), green (490-559 nm), yellow (560-589 nm), orange (590-629 nm), red (630-699 nm), and infra-red (700-800 nm) appearing in that order in FIG. 14, counted from the bottom. It is seen that light transmission decreased significantly with age for all colours though the effect of age was greatest for shorter wavelengths. For violet light, the transmission ranged from 44% in a 10 year old lens to 1% in an 80 year old lens. The transmission of red light was high at all ages, ranging from 87% in a 10 year old lens to 79% in an 80 year old lens.

FIG. 10 show model curves of transmission in the human lens at different ages (20 to 70 years of age) relative to the transmission of a 10 year old lens. The absorption curve of melanopsin is also shown to illustrate the predilection for age-related transmission loss to affect the blue end of the visible spectrum where the melanopsin photopigment is stimulated. The blue region is the part of the visible spectrum that is most affected by age. This part of the spectrum is also responsible for the stimulation of melanopsin in a subset of retinal ganglion cells. Melanopsin has an absorption peak at 480 nm. The transmission at 480 nm was 82% in a 10 year old lens, decreasing to 56% in a 40 year old lens and 23% in an 80 year old lens.

Discussion

The present example provided data enabling a modelling of the spectral transmission of the human lens as a function of age. The results are useful in various applications such as retinal light dosimetry during therapeutic light exposure or in psychophysical examinations and experimental phototherapy of winter depression.

The young lens is highly transparent to all visible wavelengths but transmission decreases drastically after the age of 30 years. Though the accumulation of yellow chromophores is responsible for the preferential loss of light transmission in the blue end of the spectrum it is not in itself a cause of loss of visual function but it reduces the ability to perceive and distinguish shades of blue.

Decreased transmission of blue light by the aged lens may be responsible for the increased risk of depression and sleep disorders seen in the elderly population through depressed activation of melanopsin. Melanopsin is a photosensitive pigment that is important for circadian photoentrainment by stimulating melatonin secretion by the pineal gland. It is expressed in a small subset of retinal ganglion cells. It has an absorption maximum near 480 nm and it is active only in bright light. Thus, dim light and a reduced transmission of blue light has a major impact on the circadian pace, sleep disorders and mental mood.

### Further Details of the Invention

The invention will now be described in more detail by means of the following sequentially numbered items:
1. A method of prevention, treatment and/or amelioration of a disease and/or disorder which is either related to a lens of an eye and/or which may benefit from the treatment of said lens, the method comprising the exposure of said lens to radiation of a wavelength substantially between 320 nm and 800 nm, thereby inducing changes in the lens and/or its constituents, comprising:
   a) focusing a treatment light source into a selected part of the lens and/or its constituents collectively or selectively where treatment is intended to occur, and
   b) emitting light from said treatment light source, and
      c1) measuring one or more types of radiation from the selected part of the lens and utilizing this measurement to decide to stop the treatment light source or to adjust at least one of the parameters: focus, intensity, wavelength, polarization, pulse length, repetition frequency, and pulse train length of said treatment light source, or
      c2) stop the treatment light source after a predetermined period of time.
2. The method of item 1, wherein the light is emitted as continuous wave radiation.
3. The method of item 1, wherein the light is emitted as pulsed wave radiation.
4. The method of any of the preceding items, wherein the diseases and/or disorders are selected from: presbyopia; cataract at all stages; opacities, brunescence or cloudiness of the lens; refractive errors; myopia; hyperopia, astigmatism and/or nuclear sclerosis.
5. The method of any of the preceding items, wherein said disease and/or disorder is presbyopia and/or cataract.
6. The method of any of the preceding items, wherein said disease and/or disorder is a mental disease and/or disorder such as depression or winter depression.
7. The method of any of the preceding items, wherein said disease and/or disorder is a sleep disorder.
8. The method of any of the preceding items, wherein said method is applied in connection with therapeutic light exposure and/or phototherapy.
9. The method of any of the preceding items, wherein the emitted light is of a wavelength between 350 nm and 600 nm.
10. The method of any of the preceding items, wherein the emitted light is of a wavelength between and including 380 nm and 540 nm.
11. The method of any of the preceding items, wherein the emitted light is of a wavelength between and including 400 nm and 500 nm.
12. The method of any of the preceding items, wherein the emitted light is of a wavelength between and including 425 nm and 465 nm.
13. The method of any of the preceding items, wherein the emitted light is of a wavelength between and including 435 nm and 455 nm.
14. The method of any of the preceding items, wherein the emitted light is of a wavelength between and including 440 nm and 450 nm.
15. The method of any of the preceding items, wherein the emitted light is of a wavelength between and including 442 nm and 447 nm.
16. The method of any of the preceding items, wherein the intensity of the treatment light beam is between 0.0001 mW/micron̂2 and 100 mW/micron̂2, such as between 0.001 mW/micron̂2 and 50 mW/micron̂2, such as between 0.01 mW/micron̂2 and 25 mW/micron̂2, such as between 0.1 mW/micron̂2 and 10 mW/micron̂2, such as between 1 mW/micron̂2 and 5 mW/micron̂2, said intensity preferably defined substantially in front of the cornea of the eye being treated.
17. The method of any of the preceding items, wherein the emitted light is monochromatic.
18. The method of any of the preceding items, wherein the emitted light is polychromatic.
19. The method of any of the preceding items, wherein radiation protection of the retina and/or the fovea is provided.
20. The method of any of the preceding items, wherein the retina and/or the fovea is protected by means of one or more beam blockers, preferably located in front of the eye in a substantially conjugate position to the fovea.
21. The method of any of the preceding items, wherein the treatment light beam is provided at an indirect angle to the eye and wherein the treatment light beam is directed towards the lens by reflection means, such as one or more mirrors.
22. The method of any of the preceding items, further comprising scanning the treatment light source, or the light emitted from the treatment light source, relative to the lens.
23. The method of any of the preceding items, wherein step c1) furthermore comprises utilizing the measurement to decide to adjust at least one of the parameters: scan velocity, size of scanned volume, scan repetitions, and scan pattern.
24. The method of any of the preceding items 18 to 23, wherein the scan pattern of the scanning comprises a meander scan and/or a discontinuous line-by-line scan and/or a continuous back-and-forth line scan and/or a spiral scan and/or circular scan.
25. The method of item any of the preceding items where at least one of said types of radiation arises due to the emission from the treatment light source.
26. The method of any of the preceding items where at least one of said types of radiation arises due to a secondary source of radiation, such as a laser.
27. The method of item 26, wherein said secondary source of radiation is combined with the treatment light beam to provide a combined beam of treatment light and secondary laser light, thereby ensuring that the combined beam is directed to said selected part of the lens.
28. The method of any of the preceding items further comprising an initialization phase prior to steps a)-c) where non-manipulative intensity is directed to the selected part of he lens and one or more types of radiation, caused by the interaction between the selected part and the non-manipulative intensity are measured and utilizing this measurement to decide not to photomanipulate the selected part or decide to proceed with photomanipulation.
29. The method of item 28, where said initialization phase is further utilized to adjust at least one of the following: focus, intensity, wavelength, polarization, pulse length, repetition frequency, pulse train length, scan velocity, size of scanned volume, scan repetitions, and scan pattern of said treatment light source.
30. The method of any of the preceding items further comprising an assessment phase after application of the said treatment light source where non-manipulative intensity is directed to the said selected part and measuring one or more types of radiation caused by the interaction between the said part and the said non-manipulative intensity and utilizing this measurement to decide to stop further treatment of said part or to resume treatment with or without adjustment of at least one of the following: focus, intensity, wavelength, polarization, pulse length, repetition frequency, pulse train length, scan velocity, size of scanned volume, scan repetitions, and scan pattern of said treatment light source.
31. The method of any of the preceding items, where the said measurement involve determining the optical signature of the said selected part comprising at least one of the following: transient characteristics arising as an effect of the treatment pulse or any characteristic that can be recorded using steady-state or time-resolved spectroscopy, Raman spectroscopy, photon-correlation spectroscopy, fluorescence spectroscopy and/or phosphorescence spectroscopy.
32. The method of any of the preceding items where the said measurement involves detection of acoustic effects recorded using non-contact sensor(s) and/or an acoustic sensor placed in direct contact with the eye or adjacent tissue.
33. The method of any of the preceding items where the said measurement, analysis and adjustment form a feed-back loop.
34. The method of any of the preceding items where the said feed-back loop operates substantially in real-time.
35. The method of any of the preceding items where the said measurement, processing of resulting data, said adjustment(s), and renewed irradiation of the said selected part occurs at least within substantially 10 seconds.
36. The method of any of the preceding items wherein the said measurement, processing of resulting data, said adjustment(s), and renewed irradiation of the said selected part occurs at least within a time period which is substantially smaller than the spontaneous movements of the eyes (saccades) and preferably shorter than 0.1 seconds.
37. The method of any of the preceding items wherein fluorescence observed along the treatment light source path in the lens is used to adjust the focal plane relative to the lens.
38. The method of any of the preceding items where a procedure is performed comprising the following steps
   a) photomanipulating said selected part of the lens
   b) detection radiation from the said selected part of the lens
   c) gradually changing energy of said photomanipulation
   d) registering when said radiation is within a defined threshold.
39. The method of any of the preceding items further comprising a verification of efficiency by measurement of radiation due to a non-manipulative intensity directed to the said selected part.
40. The method of any of the preceding items further comprising comparing values of said radiation obtained prior to treatment with the data obtained from said verification.
41. The method of any of the preceding items where the treatment light source and/or any secondary source of radiation is focused using adaptive optics.
42. The method of item 41 where the said adaptive optics further comprises the use of a deformable mirror.
43. The method of items 41 and 42 where the said adaptive optics further comprises the use of a Hartmann-Schack sensor.
44. The method of items 41 to 43 where the adaptive optics form a feedback loop.
45. The method of items 44 where the adaptive optics is guided by radiation caused by the treatment light source or a said secondary source of radiation.
46. The method of any of the preceding items where the radiation comprises at least one of the following: fluorescence, scatter, Raman scatter, reflection, phosphorescence, and bremsstrahlung.
47. The method of any of the preceding items where the measurement comprises measuring the spectral distribution and/or intensity of the radiation.
48. The method of any of the preceding items where the measurement of the radiation is followed by temporally resolved analysis.
49. The method of any of the preceding items where the at least one treatment light source is adjusted to obtain bleaching, color change, deaggregation of lens components, depolymerization of lens proteins or other constituents of the lens, or resolubilization of lens proteins or other constituents of the lens while avoiding or minimizing cavitation, mechanical effects, acoustic effects, and/or thermal effects on molecules, components, or cells that do not form a target for treatment or are outside the selected area.
50. The method of any of the preceding items where the treatment light source is adjusted to obtain molecular cleavage of specific larger molecules or macromolecular adducts, for instance lens proteins or lens protein cross-links, without damage to healthy lens proteins, cell membranes or other healthy components of the lens, and further avoiding or minimizing cavitation, mechanical effects, acoustic effects, and/or thermal effects on molecules, components, or cells that do not form a target for treatment or are outside said selected area.
51. The method of item 50 carried out according to any of the preceding items where the radiation is fluorescence while minimizing or preventing the increase in scattering.
52. The method of any of the preceding items, wherein said treatment light source is a treatment laser system.
53. The method of item 52 where said treatment laser system comprises at least one laser source based on one or more of the following: a gaseous medium, a solid state gain medium and/or a semiconductor laser (laser diode).
54. The method of any of the preceding items 52 to 53where the said treatment laser system comprises at least one laser source based on a semiconductor laser (laser diode).
55. The method according to any of the preceding items 52 to 54, wherein the treatment laser system emits light of at least one of the following wavelengths: 387 nm, 395 nm, 405 nm, 415 nm, 430 nm, 442 nm, 445 nm, 450 nm, 458 nm, 473 nm, 488 nm, 514 nm and/or 532 nm
56. The method according to any of the preceding items 52 to 55, wherein the treatment laser system comprises at least on of the following: a cw laser at 405 nm generated by frequency doubling of an 810 nm tapered diode laser; an external phase-locked doubler resonator used to generate 300 mW of light; and/or a cw 532 nm laser with a cw diode-pumped solid state second harmonic Nd:YAG laser.
57. The method of any of the preceding items 52 to 56, where the said treatment laser system further comprises at least one means controlling the state of polarization of the treatment light source.
58. The method of any of the preceding items 3 to 57, where said pulsing comprises pulsing the treatment light source with a pulse width shorter than substantially 1 second.
59. The method of any of the preceding items to 3 to 58, where said pulsing comprises pulsing the treatment light source with a pulse width shorter than substantially 500 picoseconds.
60. The method of any of the preceding items 3 to 59, where said pulsing comprises pulsing the treatment light source with a pulse width shorter than substantially 100 femto-seconds.
61. A method of any of the preceding items 3 to 60, where said pulsing comprises pulsing the treatment light source with pulse energy density lower than substantially 1 Joule per square centimeter.
62. A method of any of the preceding items 3 to 61, where said pulsing comprises pulsing the treatment light source with pulse energy density lower than substantially 10 milli-Joules per square centimeter.
63. The method of any of the preceding items, where the said treatment light source is focused to a spot of substantially 100 microns.
64. The method of any of the preceding items where the focus of the beam of the treatment light source is scanned so as to treat at least one predefined volume, said volume being of a size enabling selective targeting of the lens substance and its sub-regions without damaging adjacent healthy or unhealthy tissue.
65. The method of item to 64 wherein said volume has a cross-section seen from the instrument corresponding to the entire lens or specific parts thereof.
66. The method of item to 64 wherein said volume has a cross-section seen from the instrument of up to about 100 square millimeters.
67. The method of any of the preceding items, wherein the eye being treated is mechanically immobilized, wholly or partly, prior to and/or during the treatment, by mechanical contact with the surface of the eye or by mechanical contact to a contact lens mounted on the eye.
68. The method of any of the preceding items, wherein the movements of the eye being treated are tracked by imaging of the eye on at least one light detector.
69. The method of any of the preceding items, wherein the movement of the eye is found by tracking reference points in the eye.
70. The method of any of the preceding items further comprising a method for monitoring and/or tracking the orientation of an eye in space by simultaneously monitoring the surface or anterior part of the eye and the fundus (posterior inside of the eye) and calculating the orientation of the eye in space.
71. The method for conditioning the eye prior to treatment, during treatment, prior to assessment or during assessment by applying at least on of the following: heat, cold and magnetic field.
72. The method according to any of the preceding items wherein the lens to be treated stems from the living eye of an animal or a human being.
73. A method of treatment and/or amelioration of a presbyopic and/or cataractous disorder of a lens of an eye, the method comprising the exposure of said lens to radiation of a wavelength substantially between 350 nm and 550 nm, thereby inducing changes in the lens and/or its constituents, comprising;
   a) focusing a treatment light source into a selected part of the lens and/or its constituents collectively or selectively where treatment is intended to occur;
   b) emitting light from the treatment light source;
      c1) measuring one or more types of radiation from the said selected part and utilizing this measurement to decide to stop the said treatment light source or to adjust at least one of the parameters: focus, intensity, wavelength, polarization, pulse length, repetition frequency, and pulse train length of said treatment light source, or
      c2) stop the treatment light source after a predetermined period of time,
   thereby photomanipulating the lens and/or its constituents thus treating and/or ameliorating said presbyopic and/or cataractous disorder.
74. The method of any of the preceding items, wherein adjuvant pharmaceuticals are administered.
75. The method of item 74, wherein said pharmaceuticals quench free radicals in the eye.
76. The method of any of the preceding items further providing optimum benefit of treatment while minimizing the risk of damage consisting of controlling the patient's body fluid levels, e.g. serum or plasma values, of one or more vitamins, including riboflaving, or other physiological or extrinsic substances with the aim of excluding patients with values for riboflavin or other photosensitizing agents above a defined threshold or excluding patients with insufficient levels of substances that may provide protection against adverse effects.
77. The method of any of the preceding items further providing optimum benefit of treatment while minimizing the risk of damage consisting of applying exposure to light of duration 0.000001 second to 10000 seconds with a spectral composition ranging from white light to one or more selected spectral bands of visible light, including such band or bands that most optimally fit the absorption peak or peaks of one or more retinal photopigments, including the photopigments of the rod and the cone photoreceptors and the photopigments of the retinal ganglion cells or any combination of the photopigments of the rods, cones, ganglion cells, and other light-sensitive cells of the eye, the luminance of the said flash of light ranging from 10 candela per square meter to 1 million candela per square meter and the area of exposure including all or one or more parts of the retina, the desired effect of the light exposure comprising the bleaching of retinal photopigments such that subsequent exposure of the eye to radiation targeting conditions in the lens can be made under conditions that reduce the risk of exposing the retina to harmful types or levels of radiation.
78. The method of item 77 herein said flash illumination of the eye uses light diffusely illuminating and being reflected from a large reflecting surface placed in front of a human subject with open eyelids, the light consisting of the wavelenghts 480 nm, 500 nm, and 550 nm or any combination thereof and any extension of the bandwidths of the said spectral lines up to and including a continuous band ranging from 450 nm to 600 nm.
79. The method of any of the preceding items further providing optimum benefit of treatment while minimizing the risk of damage comprising the repeated exposure of the eye to light of the said characteristics, with optional intraoperative monitoring of light sensitivity, including the provision of visual objectives that alert the patient to respond in a manner, such as activating an electric contact, indicating that a renewed bleaching exposure is needed and prompting an operator or a machine to effect renewed bleaching.
80. The method of any of the preceding items further providing optimum benefit of treatment while minimizing the risk of damage consisting of inhibiting processes in the retina that occur in response to illumination, including but not limited to those processes that are responsible for vision and for controlling the diurnal cycle.
81. The method of any of the preceding items further providing optimum benefit of treatment while minimizing the risk of damage consisting of inhibiting the regeneration of visual pigment in the retina by pharmacologically blocking enzymes, receptors, channels genes or cells that contribute to the continuous restoration of light sensitivity in the living eye, including but not limited to methods comprising the administration of pharmaceutical agents such as fenretinide, 13-cis-retinoic acid (isotretinoin), 11-cis-retinol, 11-cis-retinal, 11-cis-retinyl bromoacetate, acitretin, etretinate, 4-oxo-isotretinoin, motretinide, retinaldehyde, all-trans-retinyl bromoacetate, all-trans-retinyl chloroacetate, and retinoyl betaglucoronide.
82. A system for prevention, treatment and/or amelioration of a disease and/or disorder which is either related to a lens of an eye and/or which may benefit from the treatment of said lens, said system comprising:
   a) focusing means for focusing a treatment light beam of a wavelength substantially between 320 nm and 800 nm into a selected part of the lens and/or its constituents collectively or selectively where treatment is intended to occur, and
   b) means for emitting and/or unblocking said treatment light beam, thereby inducing changes in the lens and/or its constituents, and
   c1) means for stopping and/or blocking said treatment light beam after a predetermined period of time, and/or
   d1) means for measuring one or more types of radiation from said selected part, and
   d2) means for processing said one or more type of radiation from said selected part, and
   d3) means for adjusting, based on at least part of the output of the means for processing, at least one of the parameters for said treatment light beam: focus, intensity, wavelength, pulse length, repetition frequency, and pulse train length of said treatment light beam.
83. The system of item 82, further comprising at least one light source for generating said treatment light beam.
84. The system of any of items 82 to 83, wherein said treatment light beam is at least partly generated by means of at least one filter.
85. The system of any of items 82 to 84, wherein said treatment light beam is at least partly generated by means of at least one laser or a laser system, such as a treatment laser system.
86. The system of any of items 82 to 85, wherein said treatment light beam is at least partly generated by means of at least one filament lamp and/or at least one incandescent lamp and/or at least one white light source.
87. The system of any of items 82 to 86, wherein said treatment light beam is at least partly generated by means of sunlight.
88. The system of any of items 82 to 87, wherein the light is emitted as continuous wave radiation.
89. The system of any of items 82 to 88, wherein the light is emitted as pulsed wave radiation.
90. The system of any of items 82 to 89, wherein the diseases and/or disorders are selected from: presbyopia; cataract at all stages; opacities, brunescence or cloudiness of the lens; refractive errors; myopia; hyperopia, astigmatism and/or nuclear sclerosis.
91. The system of any of items 82 to 90, wherein said disease and/or disorder is presbyopia and/or cataract.
92. The system of any of items 82 to 91, wherein said disease and/or disorder is a mental disease and/or disorder such as depression or winter depression.
93. The system of any of items 82 to 92, wherein said disease and/or disorder is a sleep disorder.
94. The system of any of items 82 to 93, provided in connection with therapeutic light exposure and/or phototherapy.
95. The system of any of items 82 to 94, wherein the emitted light is of a wavelength between 380 nm and 600 nm.
96. The system of any of items 82 to 94, wherein the emitted light is of a wavelength between and including 380 nm and 540 nm.
97. The system of any of items 82 to 94, wherein the emitted light is of a wavelength between and including 400 nm and 500 nm.
98. The system of any of items 82 to 94, wherein the emitted light is of a wavelength between and including 425 nm and 465 nm.
99. The system of any of items 82 to 94, wherein the emitted light is of a wavelength between and including 435 nm and 455 nm.
100. The system of any of items 82 to 94, wherein the emitted light is of a wavelength between and including 440 nm and 450 nm.
101. The system of any of items 82 to 94, wherein the emitted light is of a wavelength between and including 442 nm and 447 nm.
102. The system of any of items 82 to 101, wherein the intensity of the treatment light beam is between 0.01 mW/mm² and 1000 mW/mm², such as between 0.01 mW/mm² and 0.1 mW/mm², such as between 0.1 mW/mm² and 1 mW/mm², such as between 1 mW/mm² and 10 mW/mm², such as between 10 mW/mm² and 50 mW/mm², such as between 50 mW/mm² and 100 mW/mm², such as between 100 mW/mm² and 500 mW/mm², such as between 500 mW/mm² and 1000 mW/mm², said intensity preferably defined substantially in front of the cornea of the eye being treated.
103. The system of any of items 82 to 102, wherein the emitted light is monochromatic.
104. The system of any of items 82 to 103, wherein the emitted light is polychromatic.
105. The system of any of items 82 to 104, wherein the emitted light is polarized.
106. The system of any of items 82 to 105, wherein radiation protection of the retina and/or the fovea is provided.
107. The system of any of items 82 to 106, wherein the retina and/or the fovea is protected by means of one or more beam blockers, preferably located in front of the eye in a substantially conjugate position to the fovea.
108. The system of any of items 82 to 107, wherein the treatment light beam is provided at an indirect angle to the eye and wherein the treatment light beam is directed towards the lens by reflection means, such as one or more mirrors.
109. The system of any of items 82 to 108, furthermore comprising means for scanning the treatment light beam relative to the lens.
110. The system of any of items 82 to 109, wherein step d3) furthermore comprises utilizing the measurement to decide to adjust at least one of the parameters: scan velocity, size of scanned volume, scan repetitions, and scan pattern.
111. The system of any of items 82 to 110, furthermore comprising means for scanning the treatment light beam relative to the lens in a meander scan and/or a discontinuous line-by-line scan and/or a continuous back-and-forth line scan and/or a spiral scan and/or circular scan.
112. The system of any of items 82 to 111, where at least one of said types of radiation arises due to the emission from the treatment light beam.
113. The system of any of items 82 to 112, where at least one of said types of radiation arises due to a secondary source of radiation, such as a laser.
114. The system of item 113, wherein said secondary source of radiation is combined with the treatment light beam to provide a combined beam of treatment light and secondary laser light, thereby ensuring that the combined beam is directed to said selected part of the lens.
115. The system of any of items 82 to 114, further comprising an initialization phase prior to steps a)-d) where non-manipulative intensity is directed to the selected part of the lens and one or more types of radiation, caused by the interaction between the selected part and the non-manipulative intensity are measured and utilizing this measurement to decide not to photomanipulate the selected part or decide to proceed with photomanipulation.
116. The system of item 115, where said initialization phase is further utilized to adjust at least one of the following: focus, intensity, wavelength, polarization, pulse length, repetition frequency, pulse train length, scan velocity, size of scanned volume, scan repetitions, and scan pattern of said treatment light beam.
117. The system of any of items 82 to 116, further comprising an assessment phase after application of the said treatment light beam where non-manipulative intensity is directed to the said selected part and measuring one or more types of radiation caused by the interaction between the said part and the said non-manipulative intensity and utilizing this measurement to decide to stop further treatment of said part or to resume treatment with or without adjustment of at least one of the following: focus, intensity, wavelength, polarization, pulse length, repetition frequency, pulse train length, scan velocity, size of scanned volume, scan repetitions, and scan pattern of said treatment light beam.
118. The system of any of items 82 to 117, where the said measurement involve determining the optical signature of the said selected part comprising at least one of the following: transient characteristics arising as an effect of the treatment pulse or any characteristic that can be recorded using steady-state or time-resolved spectroscopy, Raman spectroscopy, photon-correlation spectroscopy, fluorescence spectroscopy and/or phosphorescence spectroscopy.
119. The system of any of items 82 to 118 where the said measurement involves detection of acoustic effects recorded using non-contact sensor (s) and/or an acoustic sensor placed in direct contact with the eye or adjacent tissue.
120. The system of any of items 82 to 119 where the said measurement, analysis and adjustment form a feed-back loop.
121. The system of any of items 82 to 120, where the said feed-back loop operates substantially in real-time.
122. The system of any of items 82 to 121, where the said measurement, processing of resulting data, said adjustment(s), and renewed irradiation of the said selected part occurs at least within substantially 10 seconds.
123. The system of any of items 82 to 122, wherein the said measurement, processing of resulting data, said adjustment(s), and renewed irradiation of the said selected part occurs at least within a time period which is substantially smaller than the spontaneous movements of the eyes (saccades) and preferably shorter than 0.1 seconds.
124. The system of any of items 82 to 123, wherein fluorescence observed along the treatment light beam path in the lens is used to adjust the focal plane relative to the lens.
125. The system of any of items 82 to 124, where a procedure is performed comprising the following steps
   a) photomanipulating said selected part
   b) detection radiation from the said selected part
   c) gradually changing energy of said photomanipulation
   d) registering when said radiation is within a defined threshold.
126. The system of any of items 82 to 125, further comprising a verification of efficiency by measurement of radiation due to a non-manipulative intensity directed to the said selected part.
127. The system of any of items 82 to 126, further comprising comparing values of said radiation obtained prior to treatment with the data obtained from said verification.
128. The system of any of items 82 to 127, where the treatment light beam and/or any secondary source of radiation is focused using adaptive optics.
129. The system of item 128, where the said adaptive optics further comprises the use of a deformable mirror.
130. The system of any of items 128 and 129, where the said adaptive optics further comprises the use of a Hartmann-Schack sensor.
131. The system of any of items 128 to 130, where the adaptive optics forms a feedback loop.
132. The system of item 131 where the adaptive optics is guided by radiation caused by the treatment light beam or a said secondary source of radiation.
133. The system of any of items 82 to 132, where the radiation comprises at least one of the following: fluorescence, scatter, Raman scatter, reflection, phosphorescence, and bremsstrahlung.
134. The system of any of items 82 to 133 where the measurement comprises measuring the spectral distribution and/or intensity of the radiation.
135. The system of any of items 82 to 134 where the measurement of the radiation is followed by temporally resolved analysis.
136. The system of any of items 82 to 135, where the at least one treatment light beam is adjusted to obtain bleaching, color change, deaggregation of lens components, depolymerization of lens proteins or other constituents of the lens, or resolubilization of lens proteins or other constituents of the lens while avoiding or minimizing cavitation, mechanical effects, acoustic effects, and/or thermal effects on molecules, components, or cells that do not form a target for treatment or are outside the selected area.
137. The system of any of items 82 to 136, where the treatment light beam is adjusted to obtain molecular cleavage of specific larger molecules or macromolecular adducts, for instance lens proteins or lens protein cross-links, without damage to healthy lens proteins, cell membranes or other healthy components of the lens, and further avoiding or minimizing cavitation, mechanical effects, acoustic effects, and/or thermal effects on molecules, components, or cells that do not form a target for treatment or are outside said selected area.
138. The system of item 137 carried out according to any of the preceding items where the radiation is fluorescence while minimizing or preventing the increase in scattering.
139. The system of any of items 85 to 138, where the said treatment laser system comprises at least one laser source based on one or more of the following: a gaseous medium, a solid state gain medium and/or a semiconductor laser (laser diode).
140. The system of any of items 85 to 139, where the said treatment laser system comprises at least one laser source based on a semiconductor laser (laser diode).
141. The system of any of items 85 to 140, wherein the treatment laser system emits light of at least one of the following wavelengths: 387 nm, 395 nm, 405 nm, 415 nm, 430 nm, 442 nm, 450 nm, 458 nm, 473 nm, 488 nm, 514 nm and/or 532 nm.
142. The system of any of items 85 to 141, wherein the treatment laser system comprises at least one of the following: a cw laser at 405 nm generated by frequency doubling of an 810 nm tapered diode laser; an external phase-locked doubler resonator used to generate 300 mW of light; and/or a cw 532 nm laser with a cw diode-pumped solid state second harmonic Nd:YAG laser.
143. The system of any of items 85 to 142, where the said treatment laser system further comprises at least one means for controlling the state of polarization of the treatment light beam.
144. The system of any of items 89 to 143, where said pulsing comprises pulsing the treatment light beam with a pulse width shorter than substantially 1 second.
145. The system of any of items 89 to 144, where said pulsing comprises pulsing the treatment light beam with a pulse width shorter than substantially 500 picosecond.
146. The system of any of items 89 to 145, where said pulsing comprises pulsing the treatment light beam with a pulse width shorter than substantially 100 femto-seconds.
147. The system of any of items 89 to 146 where said pulsing comprises pulsing the treatment light beam with pulse energy density lower than substantially 1 Joule per square centimetre.
148. The system of any of items 89 to 147, where said pulsing comprises pulsing the treatment light beam with pulse energy density lower than substantially 10 milli-Joules per square centimetre.
149. The system of any of items 82 to 148, where the said treatment light beam is focused to a spot of substantially 100 microns.
150. The system of any of items 82 to 149, where the focus of the treatment light beam is scanned so as to treat at least one predefined volume, said volume being of a size enabling selective targeting of the lens substance and its sub-regions without damaging adjacent healthy or unhealthy tissue.
151. The system of item 150 wherein the said volume has a cross-section seen from the instrument corresponding to the entire lens or specific parts thereof.
152. The system of any of items to 150 to 151 wherein the said volume has a cross-section seen from the instrument of up to about 100 square millimeters.
153. The system of any of items 82 to 152, wherein the eye being treated is mechanically immobilized, wholly or partly, prior to and/or during the treatment, by mechanical contact with the surface of the eye or by mechanical contact to a contact lens mounted on the eye.
154. The system of any of items 82 to 153, wherein the movements of the eye being treated are tracked by imaging of the eye on at least one light detector.
155. The system of any of items 82 to 154, wherein the movement of the eye is found by tracking reference points in the eye.
156. The system of any of items 82 to 155, further comprising a system for monitoring and/or tracking the orientation of an eye in space by simultaneously monitoring the surface or anterior part of the eye and the fundus (posterior inside of the eye) and calculating the orientation of the eye in space.
157. The system of any of items 82 to 156, for conditioning the eye prior to treatment, during treatment, prior to assessment or during assessment by applying at least on of the following: heat, cold and magnetic field.
158. The system of any of items 82 to 157, wherein the lens to be treated stems from the living eye of an animal or a human being.
159. A system for the treatment and/or amelioration of a presbyopic and/or cataractous disorder of a lens of an eye, said system comprising;
   a) focusing means for focusing a treatment light beam of a wavelength substantially between 350 nm and 550 nm into a selected part of the lens and/or its constituents collectively or selectively where treatment is intended to occur, and
   b) means for emitting and/or unblocking said treatment light beam, thereby inducing changes in the lens and/or its constituents, and
   c1) means for stopping and/or blocking said treatment light beam after a predetermined period of time, and/or
   d1) means for measuring one or more types of radiation from said selected part, and
   d2) means for processing said one or more type of radiation from said selected part, and
   d3) means for adjusting, based on at least part of the output of the means for processing, at least one of the parameters for said treatment light beam: focus, intensity, wavelength, pulse length, repetition frequency, and pulse train length of said treatment light beam,
   thereby photomanipulating the lens and/or its constituents thus treating and/or ameliorating said presbyopic and/or cataractous disorder.
160. The system of any of items 82 to 159, wherein adjuvant pharmaceuticals are administered.
161. The system of item 160, wherein said pharmaceuticals quench free radicals in the eye.
162. The system of any of items 82 to 161, further providing optimum benefit of treatment while minimizing the risk of damage consisting of controlling the patient's body fluid levels, e.g. serum or plasma values, of one or more vitamins, including riboflavin, or other physiological or extrinsic substances with the aim of excluding patients with values for riboflavin or other photosensitizing agents above a defined threshold or excluding patients with insufficient levels of substances that may provide protection against adverse effects.
163. The system of any of items 82 to 162, further providing optimum benefit of treatment while minimizing the risk of damage consisting of applying exposure to light of duration 0.000001 second to 10000 seconds with a spectral composition ranging from white light to one or more selected spectral bands of visible light, including such band or bands that most optimally fit the absorption peak or peaks of one or more retinal photopigments, including the photopigments of the rod and the cone photoreceptors and the photopigments of the retinal ganglion cells or any combination of the photopigments of the rods, cones, ganglion cells, and other light-sensitive cells of the eye, the luminance of the said flash of light ranging from 10 candela per square meter to 1 million candela per square meter and the area of exposure including all or one or more parts of the retina, the desired effect of the light exposure comprising the bleaching of retinal photopigments such that subsequent exposure of the eye to radiation targeting conditions in the lens can be made under conditions that reduce the risk of exposing the retina to harmful types or levels of radiation.
164. The system of item 163, wherein said flash illumination of the eye uses light diffusely illuminating and being reflected from a large reflecting surface placed in front of a human subject with open eyelids, the light consisting of the wavelengths 480 nm, 500 nm, and 550 nm or any combination thereof and any extension of the bandwidths of the said spectral lines up to and including a continuous band ranging from 450 nm to 600 nm.
165. The system of any of items 82 to 164, further providing optimum benefit of treatment while minimizing the risk of damage comprising the repeated exposure of the eye to light of the said characteristics, with optional intraoperative monitoring of light sensitivity, including the provision of visual objectives that alert the patient to respond in a manner, such as activating an electric contact, indicating that a renewed bleaching exposure is needed and prompting an operator or a machine to effect renewed bleaching.
166. The system of any of items 82 to 165, further providing optimum benefit of treatment while minimizing the risk of damage consisting of inhibiting processes in the retina that occur in response to illumination, including but not limited to those processes that are responsible for vision and for controlling the diurnal cycle.
167. The system of any of items 82 to 166, further providing optimum benefit of treatment while minimizing the risk of damage consisting of inhibiting the regeneration of visual pigment in the retina by pharmacologically blocking enzymes, receptors, channels, genes or cells that contribute to the continuous restoration of light sensitivity in the living eye, including but not limited to systems comprising the administration of pharmaceutical agents such as fenretinide, 13-cis-retinoic acid (isotretinoin), 11-cis-retinol, 11-cis-retinal, 11-cis-retinyl bromoacetate, acitretin, etretinate, 4-oxo-isotretinoin, motretinide, retinaldehyde, all-trans-retinyl bromoacetate, all-trans-retinyl chloroacetate, and retinoyl betaglucoronide

### REFERENCES

Benedek G B. Theory of transparency of the eye. Applied Optics 1971; 10:459-73.

Chylack L T, Jr., Wolfe J K, Singer D M et al. The Lens Opacities Classification System III. The Longitudinal Study of Cataract Study Group. Arch. Ophthalmol. 1993; 111:831-6.

Davies M J, Truscott R J. Photo-oxidation of proteins and its role in cataractogenesis. J. Photochem. Photobiol. B 2001; 63:114-25. Dillon J, Roy D, Spector A et al. UV laser photodamage to whole lenses. Exp. Eye Res. 1989;49:959-66.

Glasser, A., and M. C. W. Campbell, “Biometric, optical and physical changes in the isolated human crystalline lens with age in relation to presbyopia”, Vision Research 39, 1991-2015 (1999).

Glasser A, Croft M A, Kaufman P L. Aging of the human crystalline lens and presbyopia. Int. Ophthalmol Clin. 2001;41:1-15.

Gorgels, T. and Norren, D., “Ultraviolet and Green Light Cause Different Types of Damage in Rat Retina”, Investigative Ophthalmology & Visual Science, April 1995, Vol. 36, No. 5, 851-863

Theo G. M. F. Gorgels? and Dirk van Norren, “Ultraviolet and Green Light Cause Different Types of Damage in Rat Retina

Hankins M W, Peirson S N, Foster R G. Melanopsin: an exciting photopigment. Trends Neuro-sci 2008; 31(1):27-36.

Javitt J C, Wang F, West S K. Blindness due to cataract: Epidemiology and prevention. Annual Review of Public Health 1996; 17:159-77.

Kessel, L., S. Kalinin, V. Soroka, M. Larsen, and L. B. A. Johansson, “Impact of UVR-A on whole human lenses, supernatants of buffered human lens homogenates, and purified argpyrimidine and 3-OH-kynurenine”, Acta Ophthalmologica Scandinavica 83, 221-227 (2005).

Lynnerup, N., H. Kjeldsen, S. Heegaard, C. Jacobsen, and J. Heinemeier, “Radiocarbon Dating of the Human Eye Lens Crystallines Reveal Proteins without Carbon Turnover throughout Life”, PLoS. ONE. 3, e1529 (2008).

Masaki H, Okano Y, Sakurai H. Generation of active oxygen species from advanced glycation end-products (AGEs) during ultraviolet light A (UVA) irradiation and a possible mechanism for cell damaging. Biochimica et Biophysica Acta-General Subjects 1999; 1428:45-56.

Monnier V M, Cerami A. Nonenzymatic browning in vivo: possible process for aging of long-lived proteins. Science 1981; 211:491-3.

Ponce A, Sorensen C, Takemoto L. Role of short-range protein interactions in lens opacifications. Molecular Vision 2006; 12:879-84.

1. A system for prevention, treatment and/or amelioration of a disease and/or disorder which is either related to a lens of an eye and/or which may benefit from the treatment of said lens, said system comprising:
a) focusing means for focusing a treatment light beam of continuous wave light or pulsed light with a pulse width in the long range, such as in the second, millisecond or microsecond range, of a wavelength between 380 nm and 540 nm into a selected part of the lens and/or its constituents collectively or selectively where treatment is intended to occur, the intensity of the treatment light beam, substantially in front of the cornea of the eye being treated, is between 0.01 mW/mm² and 500 mW/mm², and
b) means for emitting and/or unblocking said treatment light beam, thereby inducing changes in the lens and/or its constituents, and
c1) means for stopping and/or blocking said treatment light beam after a predetermined period of time, and/or
d1) means for measuring one or more types of radiation from said selected part, and
d2) means for processing said one or more type of radiation from said selected part, and
d3) means for adjusting, based on at least part of the output of the means for processing, at least one of the parameters for said treatment light beam: focus, intensity, wavelength, pulse length, repetition frequency, and pulse train length of said treatment light beam.

2. The system of claim 1 wherein the wavelength of the treatment light beam is between 375 and 520 nm.

3. The system of claim 1, wherein said treatment light beam is at least partly generated by means of at least one laser or a laser system, said laser emitting continuous wave light of a wavelength between 380 nm and 540 nm.

4. The system of claim 1, wherein the diseases and/or disorders are selected from: presbyopia; cataract at all stages; opacities, brunescence or cloudiness of the lens; refractive errors; myopia; hyperopia, astigmatism, nuclear sclerosis and/or a mental disease.

5. The system of claim 1, furthermore comprising means for scanning the treatment light beam relative to the lens.

6. The system of claim 1 wherein the retina and/or the fovea of eye under treatment is protected by means of one or more beam blockers located in front of the eye in a substantially conjugate position to the fovea.

7. The system of claim 1, where at least one of said types of radiation arises due to the emission from the treatment light beam, and/or due to a secondary source of radiation.

8. The system of claim 7, wherein said secondary source of radiation is combined with the treatment light beam to provide a combined beam of treatment light and secondary laser light, thereby ensuring that the combined beam is directed to said selected part of the lens.

9. The system of claim 1, wherein said system operates in an initialization phase prior to steps a)-d) where non-manipulative intensity is directed to the selected part of the lens and one or more types of radiation, caused by the interaction between the selected part and the non-manipulative intensity are measured and utilizing this measurement to decide not to photomanipulate the selected part or decide to proceed with photomanipulation.

10. The system of claim 1, where said initialization phase is further utilized to adjust at least one of the following: focus, intensity, wavelength, polarization, pulse length, repetition frequency, pulse train length, scan velocity, size of scanned volume, scan repetitions, and scan pattern of said treatment light beam.

11. The system of claim 1, where the said measurement involve determining the optical signature of the said selected part comprising at least one of the following: transient characteristics arising as an effect of the treatment pulse or any characteristic that can be recorded using steady-state or time-resolved spectroscopy, Raman spectroscopy, photon-correlation spectroscopy, fluorescence spectroscopy and phosphorescence spectroscopy.

12. The system of claim 1, where the said measurement, analysis and adjustment form a feed-back loop.

13. The system of claim 1, wherein the said measurement, processing of resulting data, said adjustment(s), and renewed irradiation of the said selected part occurs at least within a time period which is substantially smaller than the spontaneous movements of the eyes (saccades).

14. The system of claim 1, further comprising:
means for carrying out a verification of efficiency by measurement of radiation due to a non-manipulative intensity directed to the said selected part, and
means for comparing values of said radiation obtained prior to treatment with the data obtained from said verification.

15. The system of claim 1, where the radiation comprises at least one of the following: fluorescence, scatter, Raman scatter, reflection, transmission, phosphorescence, and bremsstrahlung.

16. The system of claim 1, where the at least one treatment light beam is adjusted to obtain bleaching, color change, deaggregation of lens components, depolymerization of lens proteins or other constituents of the lens, or resolubilization of lens proteins or other constituents of the lens while avoiding or minimizing cavitation, mechanical effects, acoustic effects, and/or thermal effects on molecules, components, or cells that do not form a target for treatment or are outside the selected area.

17. The system of claim 1, where the treatment light beam is adjusted to obtain molecular cleavage of specific larger molecules or macromolecular adducts without damage to healthy lens proteins, cell membranes or other healthy components of the lens, and further avoiding or minimizing cavitation, mechanical effects, acoustic effects, and/or thermal effects on molecules, components, or cells that do not form a target for treatment or are outside said selected area.

18. The system of claim 1, wherein the eye being treated is mechanically immobilized, wholly or partly, prior to and/or during the treatment, by mechanical contact with the surface of the eye or by mechanical contact to a contact lens mounted on said eye.

19. The system of claim 1, wherein a contact lens mounted on the eye being treated is at least a part of said focusing means for focusing the treatment light beam.

20. The system of claim 1, including means for administering adjuvant pharmaceuticals to the eye.

21. A method of prevention, treatment and/or amelioration of a disease and/or disorder which is either related to a lens of an eye and/or which may benefit from the treatment of said lens, the method comprising the exposure of said lens to radiation of a wavelength substantially between 320 rim and 800 nm, thereby inducing changes in the lens and/or its constituents, comprising:
a) focusing a treatment light source into a selected part of the lens and/or its constituents collectively or selectively where treatment is intended to occur, and
b) emitting light from said treatment light source, and
c1) measuring one or more types of radiation from the selected part of the lens and utilizing this measurement to decide to stop the treatment light source or to adjust at least one of the parameters: focus, intensity, wavelength, polarization, pulse length, repetition frequency, and pulse train length of said treatment light source, or
c2) step stopping the treatment light source after a predetermined period of time.

22. The method of claim 21, wherein said treatment light beam is at least partly generated by means of at least one laser, said laser emitting continuous wave light of a wavelength between 380 nm and 540 nm.

23. The method of claim 21, wherein the diseases and/or disorders are selected from: presbyopia; cataract at all stages;
opacities, brunescence or cloudiness of the lens; refractive errors; myopia; hyperopia, astigmatism, nuclear sclerosis and/or a mental disease.
